(19) United States
(12) Patent Application Publication
(10) Pub. No.: US 2013/0085095 A1
(43) Pub. Date: Apr. 4, 2013

(54) PROTEIN COMPLEMENTATION REGULATORS

(71) Applicant: Trustees of Boston University, Boston, MA (US)

(72) Inventor: Paul Terence Toran, Allston, MA (US)

(73) Assignee: Trustees of Boston University, Boston, MA (US)

(21) Appl. No.: 13/631,200

(22) Filed: Sep. 28, 2012

Related U.S. Application Data
(60) Provisional application No. 61/541,003, filed on Sep. 29, 2011.

(51) Int. Cl. C12N 15/115

(52) U.S. Cl. CPC C12N 15/115
USPC 514/1.1; 530/300; 536/23.1; 435/6.12

(57) ABSTRACT

The present invention relates to protein complementation regulators and methods for reducing target-independent interaction of protein complementation molecules.

### CROSS-REFERENCE TO RELATED APPLICATIONS

This application claims the priority benefit of U.S. Provisional Application Ser. No. 61/541,003, filed Sep. 29, 2011; which is hereby incorporated by reference in its entirety.

### TECHNICAL FIELD

The present invention relates to protein complementation regulators and methods for reducing target-independent interaction of protein complementation molecules.

### BACKGROUND

Protein complementation is a comparatively new method whereby a protein is split into two or more inactive fragments which can reassemble to form an active protein. This method has various applications such as detection of biomolecular interactions in vivo or in vitro by using split protein fragments having a detectable signal. Alternatively, this method can be used to deliver a split toxin to cells expressing a specific target. Presence of this target allows the split toxin to assemble, thereby killing the target cell.

Traditional methods of detecting target molecules, such as using RNA-binding proteins or oligonucleotide probes for RNA fused to a fluorescent molecule, have severe drawbacks and limitations. A substantial limitation is the high background signal of the fully functional fluorescent protein fused to the RNA-binding protein making actual monitoring difficult. In bacteria there is also a high background fluorescence caused by the full-length fluorescent protein, which tends to aggregate and the aggregates can be confused with the RNA-protein complexes. In eukaryotes, separation of the fluorescent protein and RNP complexes in different compartments helps in some cases (Shav-Tal et al., EMBO J., 25(15):3469-79 (2006)), but generally high fluorescent background attributed to the full-length fluorescent protein limits sensitivity of this approach.

Further, problems with sensitivity can arise by the low concentration of a target within the cell. In most cases only highly abundant targets can be detected. Another obstacle of using oligonucleotide probes for RNA detection in vivo is their fast accumulation in the nucleus.

Methods for killing target cells by selectively delivering toxins via immunotoxins, have also significant limitations such as high toxicity and high immunogenicity. High toxicity is due to the use of an entire toxin linked to the delivering antibody. Since the specificity of the immunotoxin is determined by the distribution, localization and expression of the targeted antigens, non-specific binding and side-effects can occur, when the target receptors are also presented in non-target cells as well as target cells. High immunogenicity is due to the regeneration of antibodies against the toxin, which circulates unprotected in the bloodstream before it is delivered to target cells by the delivering antibody.

Various protein complementation methods have been previously described which utilize, for example, WO2004/033485, WO2007/050979, and WO2007/051002. The use of split molecular conjugates for treatment of diseases is disclosed in WO2008/133709. These split molecular conjugates target specific nucleic acids or polypeptides and form an entire toxin only in the presence of the target, thereby killing the target cell.

The disclosures of all publications, patents, patent applications and published patent applications referred to herein by an identifying citation are hereby incorporated herein by reference in their entirety.

### SUMMARY OF INVENTION

Provided herein are protein complementation regulators, wherein the protein complementation regulator reduces the ability of a first complementation molecule comprising a first probe portion and a first effector portion and a second complementation molecule comprising a second probe portion and a second effector portion to interact in the absence of binding to a target, and wherein the protein complementation regulator allows at least one probe portion to bind the target and upon binding of the target, the effector portions of the complementation molecules are capable of forming an assembled complementation complex.

Further provided herein are methods for the detection of a target comprising allowing a protein complementation regulator to reduce the ability of a first complementation molecule comprising a first probe portion and a first effector portion and the second complementation molecule comprising a second probe portion and a second effector portion to interact in the absence of target, and wherein the protein complementation regulator allows at least one probe portion to bind the target and upon binding of the target, the effector portions of the complementation molecules form an assembled complementation complex.

Provided herein methods for the detection of a target, comprising: a) reacting components under conditions that permit formation of an assembled complementation complex, said components comprising: (i) a first complementation molecule comprising a first probe portion and a first effector portion, (ii) a second complementation molecule comprising a second probe portion and a second effector portion, and (iii) a protein complementation regulator, wherein at least one of the probe portions binds to the target and upon binding to the target, the effector portions of the complementation molecules form an assembled complementation complex, wherein the protein complementation regulator reduces the ability of the first complementation molecule and the second complementation molecule to interact in the absence of target, and wherein the protein complementation regulator allows at least one probe portion to bind the target and upon binding of the target, the effector portions of the complementation molecules form an assembled complementation complex; and b) determining if an assembled complementation complex is formed.

Also provided herein are methods for reducing target binding-independent interaction of protein complementation molecules comprising allowing a protein complementation regulator to reduce the ability of a first complementation molecule comprising a first probe portion and a first effector portion and the second complementation molecule comprising a second probe portion and a second effector portion to interact in the absence of target, and wherein the protein complementation regulator allows at least one probe portion to bind the target and upon binding of the target, the effector portions of the complementation molecules form an assembled complementation complex.

Methods for reducing target binding-independent interaction of protein complementation molecules are also provided herein, comprising: a) providing: (i) a first complementation molecule comprising a first probe portion and a first effector portion, (ii) a second complementation molecule comprising a second probe portion and a second effector portion, and (iii) a protein complementation regulator, wherein at least one of the probe portions binds to the target and upon binding to the target, the effector portions of the complementation molecules form an assembled complementation complex, wherein the protein complementation regulator reduces the ability of the first complementation molecule and the second complementation molecule to interact in the absence of binding to the target, and wherein the protein complementation regulator allows at least one probe portion to bind the target and upon binding of the target, the effector portions of the complementation molecules form an assembled complementation complex; and b) allowing the components to react under conditions that permit the formation of an assembled complementation complex. In some embodiments, the methods are for reducing nonspecific, target binding-independent interactions.

Further provided herein are methods for treating and/or preventing a disease or disorder, comprising allowing a protein complementation regulator to reduce the ability of a first complementation molecule comprising a first probe portion and a first effector portion and the second complementation molecule comprising a second probe portion and a second effector portion to interact in the absence of target, and wherein the protein complementation regulator allows at least one probe portion to bind the target and upon binding of the target, the effector portions of the complementation molecules form an assembled complementation complex thereby treating and/or preventing the disease or disorder.

Provided herein are methods for treating and/or preventing a disease or disorder, comprising: a) providing: (i) an effective amount of a first complementation molecule comprising a first probe portion and a first effector portion, (ii) an effective amount of a second complementation molecule comprising a second probe portion and a second effector portion, and (iii) an effective amount of a protein complementation regulator, wherein at least one of the probe portions binds to a target associated with the disease or disorder and upon binding to the target, the effector portions of the complementation molecules form an assembled complementation complex, wherein the protein complementation regulator reduces the ability of the first complementation molecule and the second complementation molecule to interact in the absence of binding to the target, and wherein the protein complementation regulator allows at least one probe portion to bind the target and upon binding of the target, the effector portions of the complementation molecules form an assembled complementation complex; b) allowing the components to react under conditions that permit the formation of an assembled complementation complex thereby treating and/or preventing the disease or disorder.

Kits are also provided herein comprising a protein complementation regulator, wherein the protein complementation regulator reduces the ability of the first complementation molecule comprising a first probe portion and a first effector portion and a second complementation molecule comprising a second probe portion and a second effector portion to interact in the absence of binding to a target, and wherein the protein complementation regulator allows at least one probe portion to bind the target and upon binding of the target, the effector portions of the complementation molecules are capable of forming an assembled complementation complex.

In some embodiments of any of the protein complementation regulators, methods, or kits described herein, the protein complementation regulator reduces the ability of the first complementation molecule comprising the first probe portion and the first effector portion and the second complementation molecule comprising the second probe portion and the second effector portion to interact upon binding of at least one probe portion to a target compared to the ability of the first and second complementation molecules to interact in the absence of a protein complementation regulator

In some embodiments of any of the protein complementation regulators, methods, or kits described herein, the protein complementation regulator is a polypeptide or polynucleotide. In some embodiments, the protein complementation regulator is a polynucleotide. In some embodiments, the polynucleotide is RNA or DNA. In some embodiments, the protein complementation regulator is a polypeptide. In some embodiments, the polypeptide is an antibody or polypeptide aptamer.

In some embodiments of any of the protein complementation regulators, methods, or kits described herein, the protein complementation regulator is coupled to one or more of the complementation molecules. In some embodiments, the protein complementation regulator is conjugated via a covalent linkage to one or more complementation molecules. In some embodiments, the protein complementation regulator is 5′ or N-terminal to the first probe portion, between the first and second probe portion, or 3′ or C-terminal to the second probe portion. In some embodiments, the protein complementation regulator is between the first effector portion and second effector portion. In some embodiments, the protein complementation regulator is a polynucleotide less than about 200 bases or base pairs in length.

In some embodiments of any of the protein complementation regulators, methods, or kits described herein, the protein complementation regulator reduces or inhibits tertiary interaction of the complementation molecules in the absence of binding of at least one complementation molecule to the target. In some embodiments of any of the protein complementation regulators, methods, or kits described herein, the protein complementation regulator reduces or inhibits formation of the assembled complementation complex in the absence of binding of at least one complementation molecule to the target. In some embodiments of any of the protein complementation regulators, methods, or kits described herein, the protein complementation regulator destabilizes formation of the assembled complementation complex in the absence of binding of at least one complementation molecule to the target.

In some embodiments of any of the protein complementation regulators, methods, or kits described herein, both the first and second probe portions bind the target. In some embodiments, the target is a target nucleic acid or target polypeptide. In some embodiments, the target is a target nucleic acid and the target nucleic acid is RNA or DNA. In some embodiments, the target is a target nucleic acid and the target nucleic acid is associated with a disease or disorder. In some embodiments, the target is a target nucleic acid and the target nucleic acid is single-stranded or double-stranded. In some embodiments, the target nucleic acid is detected in vivo or in vitro.

In some embodiments of any of the protein complementation regulators, methods, or kits described herein, the probe portion is a nucleic acid binding motif. In some embodiments, the nucleic acid binding motif is a nucleic acid binding polypeptide or a polynucleotide. In some embodiments, the first and the second probes bind to two adjacent sequences in the target nucleic acid. In some embodiments, the first and the second probes bind to the same sequence in the target nucleic acid.

In some embodiments of any of the protein complementation regulators, methods, or kits described herein, the target is one or more target polypeptides. In some embodiments, the one or more target polypeptides are a target multimer. In some embodiments, the multimer is a dimer, a trimer or a tetramer. In some embodiments, the target polypeptide is detected in vivo or in vitro. In some embodiments, the target polypeptide is associated with a disease or disorder.

In some embodiments of any of the protein complementation regulators, methods, or kits described herein, the effector portion is an effector molecule or a molecule which directly or indirectly binds an effector molecule. In some embodiments, the effector molecule is a fluorophore, a toxin, or a polypeptide.

The protein complementation regulator, method, or kit of claim 35, wherein the molecule which directly or indirectly binds the effector molecule is a polypeptide or polynucleotide. In some embodiments, the polynucleotide is an aptamer. In some embodiments, the aptamer binds eIF4a or a fragment thereof conjugated via a covalent linkage to an effector molecule. In some embodiments, the effector molecule is a split polypeptide.

### BRIEF DESCRIPTION OF THE FIGURES

FIG. 1 shows the FACS histogram of live E. coli cells expressing different combinations of split proteins, protein complementation regulator and target RNAs. All cells were measured at equal optical densities. Cells that contained split protein expression vector but were not induced with IPTG (A). Cells that contain split fluorescent proteins and protein complementation regulator with two probe-half aptamers (P1-A1-R-A2-P2). (B). Cells that contain split fluorescent proteins, P1-A1-R-A2-P2 and target RNA with scrambled target sequence (C). Cells that contain split fluorescent proteins, P1-A1-R-A2-P2 and target RNA (D).

FIG. 2 shows microscopy images of live E. coli cells. Images of live E. coli cells taken at equal optical densities using a Nikon inverted microscope Eclipse Ti-E with a Nikon CF160 optical system at 150X. Cells that contain split fluorescent proteins and protein complementation regulator with two probe-half aptamers (P1-A1-R-A2-P2) (Row A). Cells that contain split fluorescent proteins, P1-A1-R-A2-P2 and target mRNA (Row B). Cells that contain split fluorescent proteins, P1-A1-R-A2-P2 and target mRNA with scrambled target sequence. Bright field images (Column 1). Fluorescent images (Column 2).

FIG. 3 shows the FACS histogram of live E. coli cells showing ability of protein complementation regulator to reduce background of spurious protein complementation in protein complementation assays. All cells were measured under equal optical densities. Wild-type cells (A). Cells that contain split fluorescent proteins and protein complementation regulator with two probe-half aptamers (P1-A 1-R-A2-P2) (B). Cells with split fluorescent proteins alone (C). Cells with split fluorescent proteins and protein complementation regulator with one probe-half aptamers (P1-A1-R) (D). Cells with split fluorescent proteins and protein complementation regulator without probe or aptamer sequences (R) (E). Cells that contain only full (un-split) fluorescent proteins (F).

FIG. 4 shows real-time PCR analysis of protein complementation regulator with two probe-half aptamers (P1-A1-R-A2-P2) normalized to 16S rRNA levels. Relative level of P1-A1-R-A2-P2 in cells that express split fusion proteins and P1-A1-R-A2-P2 only (A) (Mean Ct value 5S: 9.31 Aptamer Probes: 9.78), in cells that express split proteins, P1-A1-R-A2-P2 and target RNA (B) (Mean Ct value 5S: 13.46 P1-A1-R-A2-P2: 8.71), and in cells that express split proteins, P1-A1-R-A2-P2 and scrambled target RNA (C) (Mean Ct value 5S: 9.78 P1-A1-R-A2-P2: 9.61).

FIG. 5 shows real-time PCR analysis of target RNAs normalized to 16S rRNA levels. Relative level of target RNA in cells that express split fusion proteins and P1-A1-R-A2-P2 only (A) (Mean Ct value 5S: 9.38 Target RNA: 33.56), in cells that express split proteins, P1-A1-R-A2-P2 and target RNA (C) (Mean Ct value 5S: 10.17 Target RNA: 13.30), and in cells that express split proteins, P1-A1-R-A2-P2 and scrambled target RNA (D) (Mean Ct value 5S: 12.40 Scrambled Target RNA: 12.67). B, D, F are the no reverse transcriptase controls for A, C and E respectively.

FIG. 6 shows the mFOLD structure of P1-A 1-R-A2-P2.

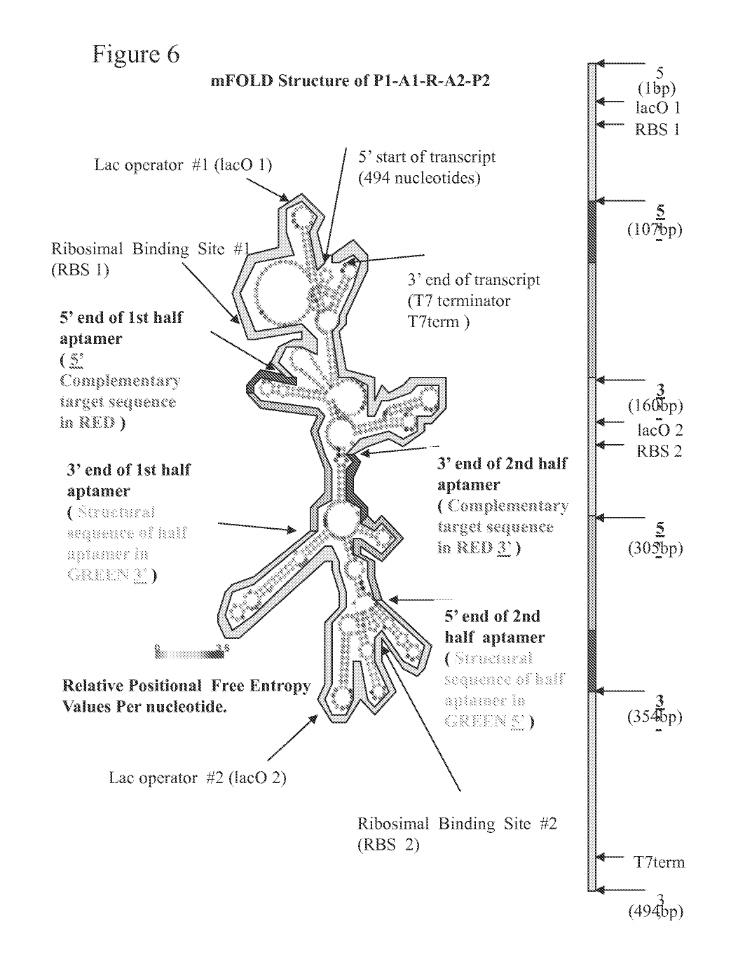

### DETAILED DESCRIPTION

The present application provides protein complementation regulators and methods of using same. For example, provided herein are methods for detecting a target in vitro or in vivo and/or treating diseases associated with a target in isolated cells or in an organism using protein complementation regulators. Specifically, a protein complementation regulator and two or more complementation molecules each comprising a probe portion and an effector portion are provided. The protein complement regulator reduces the ability of the complementation molecules to interact in the absence of specific binding to a target and allows at least one probe portion to specifically bind the target, and upon binding of the target, the effector portions of the complementation molecules form an assembled complementation complex. Although the methods described herein focus on use of two complementation molecules, it is to be understood that the methods can use more than two complementation molecules or more than two target nucleic acids, such as two or more complementation molecules or two or more target nucleic acids.

As used herein, components may include one or more complementation molecules, protein complementation regulator, and/or target.

### DEFINITIONS

The term “complementation molecule” refers to a molecule comprising a probe portion and an effector portion. The term “complementation” used herein refers to the fact that the molecules are capable of interacting with each other such as assembling to form a complex. The probe portion is responsible for binding to the target, while the effector portions upon binding of the probe portions are brought into proximity and form an assembled complex, directly, wherein the effector portion is the effector molecule or indirectly, for example, by recruiting an effector molecule to form an assembled complex. The complementation molecule can be a single type of molecule (such as a complementation molecule comprising a probe portion and an effector portion) or be a chimeric biomolecular molecule such as a conjugate comprising a probe portion conjugated to an effector portion.

The term “probe portion” refers to a portion of the complementation molecule that is capable of binding to a target.

The term “effector portion” refers to a portion of the complementation molecule that is capable of reassembly or protein complementation to form a functionally active complex upon binding to a target via the probe portion. As described herein, the effector portion can comprise an effector molecule (e.g., in active, inactive, or precursor form) or the effector portion can comprise molecules, which are capable of directly or indirectly recruiting one or more effector molecules (e.g., in active, inactive, or precursor form).

The term “effector molecule” refers to a molecule or a fragment thereof which when two or more effector molecule(s) are brought into proximity, function and have a desired effect.

The term “protein complementation regulator” refers to a molecule which is capable of reducing the ability of one or more complementation molecule(s) to interact in the absence of specific binding to a target (e.g., in the absence of a target).

The terms “specifically bind” or “binding with specificity” refers to the discriminative association of at least one of the probe portions of a complementation molecule or the discriminative collective association of probe portions of the complementation molecules with a target, for example, reacts or associates more frequently, more rapidly, with greater duration and/or with greater affinity to a target than it does with a non-target. “Specific” includes preferential association to a target compared to a non-target.

The term “specifically hybridizes” refers to the association between two single-stranded nucleic acid molecules of sufficiently complementary sequence to permit such hybridization under pre-determined conditions generally used in the art (sometimes termed “substantially complementary”). The term also refers to hybridization of an oligonucleotide with a substantially complementary sequence contained within a single-stranded DNA or RNA nucleic acid binding motif, to the substantial exclusion of hybridization of the oligonucleotide with single-stranded nucleic acids of non-complementary sequence.

The term “conjugate” or “conjugated” refers to the attachment of two or more molecules (e.g., nucleic acid, polypeptide, and/or co-factor) joined together to form one entity. The molecules (e.g., proteins and/or nucleic acids) may be attached together by linkers, chemical modification, peptide linkers, chemical linkers, covalent or non-covalent bonds, or protein fusion or by any means known to one skilled in the art. The joining may be permanent or reversible.

The term “linker” refers to a moiety which joins two or more molecules (e.g., proteins and/or nucleic acids) by means other than the production of a fusion protein or a fusion nucleic acid. A linker can be a covalent linker or a non-covalent linker Examples of covalent linkers include covalent bonds or a linker moiety covalently attached to one or more of the proteins to be linked. The linker can also be a non-covalent bond, e.g., an organometallic bond through a metal center such as platinum atom.

The term “nucleic acid” refers to deoxyribonucleotides or ribonucleotides and polymers thereof in either single- or double-stranded form. The term nucleic acid is used interchangeably with gene, cDNA, and mRNA encoded by a gene.

The terms “polypeptide” and “peptide” refer to a polymer of amino acid residues. The terms include amino acid polymers in which one or more amino acid residue is an artificial chemical analog of a corresponding naturally occurring amino acid, as well as to naturally occurring amino acid polymers.

The term “mutation” or “polymorphism” refers to a change in the nucleic acid sequence of nucleic acid, which can or can not affect the expression of the nucleic acid sequence. The term polymorphism is intended to include all polymorphisms, including deletions, substitutions, insertions, rearrangements, translocations, alternative slicing, single nucleic acid polymorphisms (SNPs), etc.

The term “recombinant” when used in reference to, for example, a cell, or nucleic acid, or vector, indicates that the cell, or nucleic acid, or vector, has been modified by the introduction of a heterologous nucleic acid or the alteration of a native nucleic acid, or that the cell is derived from a cell so modified. Thus, for example, recombinant cells express genes that are not found within the native (non-recombinant) form of the cell or express native genes that are otherwise abnormally expressed, under expressed or not expressed at all.

The term “transfer” refers to the introduction of material such as a heterologous polynucleotide, nucleic acid sequence, or fragment thereof into a host cell, using any known method in the art, for example, but not limited to direct uptake, transformation, transfection, or transduction.

The terms, “pathogenic nucleic acid” or “pathogenic DNA”, as used herein refer to the nucleic acid sequence that contributes, wholly or in part, to the symptoms, for example the structural and functional changes in cell, tissues and organs, which directly or indirectly contribute to the disease disorder or malignancy.

As used herein, “treatment” or “treating” is an approach for obtaining beneficial or desired results including clinical results. Beneficial or desired clinical results include, but are not limited to, one or more of the following: decreasing one or more symptoms resulting from the disease, diminishing the extent of the disease, stabilizing the disease (e.g., preventing or delaying the worsening of the disease), delay or slowing the progression of the disease, ameliorating the disease state, decreasing the dose of one or more other medications required to treat the disease, and/or increasing the quality of life.

As used herein, “delaying” the progression of the disease means to defer, hinder, slow, retard, stabilize, and/or postpone development of the disease. This delay can be of varying lengths of time, depending on the history of the disease and/or individual being treated.

The terms “sensitize” or “sensitizes” are used interchangeably herein and refer to making the cell sensitive, or susceptible to other secondary agents, for example other prodrugs or other environmental effects such as radiation etc.

“Prophylaxis,” “prophylactic treatment,” or “preventive treatment” refers to prevention or amelioration of the occurrence of one or more symptoms and/or their underlying cause, for example, prevention or amelioration of a disease or condition in an individual susceptible to developing a disease or condition (e.g., at a higher risk, as a result of genetic predisposition, environmental factors, predisposing diseases or disorders, or the like).

The expression “effective amount” refers to an amount of the protein complementation regulator and/or one or more complementation molecules that is effective for preventing, treating, and/or delaying the progression of the disease.

The terms, “disorder” or “disease”, are used interchangeably, refers to any alteration in the state of the body or one of its organs, interrupting or disturbing the performance of and organ function (i.e. causes organ dysfunction) and/or causing a symptom such as discomfort, dysfunction, distress, or even death to an individual afflicted with the disease. In some embodiments, symptoms such as discomfort, dysfunction, distress, or even death can occur to individuals in contact with the individual with the disease. A disease can also relate to ailing, ailment, malady, disorder, sickness, illness, complaint, and indisposition.

As used herein, the term “individual” is intended to include human and non-human animals. The term “non-human animals” includes invertebrates and vertebrates, e.g., mammals, non-mammals, such as non-human primates, horses, sheep, dogs, cows, chickens, amphibians, reptiles, rodents, etc. In certain embodiments, the individual is a mammal, e.g., a primate or a human. In some embodiments, a mammal is a human.

Reference to “about” a value or parameter includes (and describes) variations that are directed to that value or parameter per se. For example, description referring to “about X” includes description of “X”.

As used herein, the singular forms “a,” “or,” and “the” include plural referents unless the context clearly dictates otherwise.

It is understood that aspects and variations include “consisting” and/or “consisting essentially of” aspects and variations.

### Protein Complementation Regulators

A protein complementation regulator reduces the ability of one or more complementation molecules to interact in the absence of specific binding to a target. In some embodiments, the protein complementation regulator allows one or more probe portions to specifically bind the target and upon binding of the target, the effector portions of the complementation molecules are capable of forming an assembled complementation complex.

In some embodiments, the protein complementation regulator reduces the ability of one or more complementation molecules to interact in the absence of a specific target.

In some embodiments, the protein complementation regulator is associated with one or more of the complementation molecules. In some embodiment, the protein complementation regulator is coupled with one or more of the complementation molecules. In some embodiments, the protein complementation regulator is conjugated to one or more complementation molecules. In some embodiments, the protein complementation regulator is covalently conjugated to one or more complementation molecules. In some embodiments, the protein complementation regulator is non-covalently conjugated to one or more complementation molecules. In some embodiments, the protein complementation regulator is covalently linked in cis to the complementation molecule and is 5′ or N-terminal to the first probe portion, between the first probe portion and second probe portion, or 3′ or C-terminal to the second probe portion. In some embodiments, the protein complementation regulator is between the first probe portion and first effector portion and/or second probe portion and second effector portion. In some embodiments, the protein complementation regulator is covalently linked in cis to the complementation molecule and is between the first effector portion and the second effector portion.

In some embodiments, the protein complementation regulator reduces or inhibits tertiary interaction of one or more complementation molecules in the absence of binding of at least one complementation molecule to the target. In some embodiments, the protein complementation regulator, the protein complementation regulator reduces or inhibits formation of the assembled complementation complex in the absence of binding of at least one complementation molecule to the target. In some embodiments, the protein complementation regulator destabilizes formation of the assembled complementation complex in the absence of binding of at least one complementation molecule to the target.

In some embodiments, the protein complementation regulator is a nucleic acid or a polypeptide. In some embodiments, when the protein complementation regulator is a nucleic acid, the protein complementation regulator is about 200 or less bases in length. In some embodiments, the protein complementation regulator is about 175 or less bases in length. In some embodiments, the protein complementation regulator is about 150 or less bases in length. In some embodiments, the protein complementation regulator is about 100 or less bases in length. In some embodiments, the protein complementation regulator is between about any one of 10-200, 10-175, 20-175, 30-175, 40-175, 50-175, 75-175, 100-175, 125-175, 50-200, 125-175, 50-75, 75-125, 100-125, or 75-100 bases in length. In some embodiments, the protein complementation regulator is about any one of 200, 190, 180, 170, 160, 150, 140, 130, 120, 110, 100, 90, 80, 70, 60, 50, 40, 30, 20 or 10 bases in length. In some embodiments, when the protein complementation regulator is a polypeptide, the protein complementation regulator is 50 or less amino acids in length. In some embodiments, the protein complementation regulator is 30 or less amino acids in length. In some embodiments, the protein complementation regulator is between about any one of 15-50, 20-50, 30-50, 40-50, 15-40, 15-30, 15-20, 20-40, 20-30, or 30-40 amino acids in length. In some embodiments, the protein complementation regulator is about any one of 15, 20, 25, 30, 35, 40, 45 or 50 amino acids in length.

In some embodiments, the protein complementation regulator is a polynucleotide of about 175 or less bases in length is covalently linked in cis to the complementation molecule and is between the first effector portion and the second effector portion. In some embodiments, the protein complementation regulator is a polynucleotide of about 150 or less bases in length, is covalently linked in cis to the complementation molecule and is between the first effector portion and the second effector portion.

Protein complementation regulators can be identified by methods known in the art and described herein. Provided herein are methods of screening for protein complementation regulator sequences which are suitable for the compositions and methods described herein. For example, protein complementation regulators can be identified from a library of sequences. This library of potential protein complementation regulator sequences can be introducing into suitable cells together with an inducible target and complementation molecules which bind to the induced target with requisite specificity, thereby bringing the effector portions comprising fragments of a split fluorescent protein or ligands binding thereto into proximity and forming an assembled fluorescent complementation complex. The transformed cells can then be screened by FACS or fluorescence microscopy before and after inducing the target. Cells that are highly fluorescent without the target being induced are not selected and/or discarded. Cells that are highly fluorescent after the target has been induced are selected as potential protein complementation regulators. The effectiveness of the potential protein complementation regulators in inhibiting and/or reducing target independent interaction of the protein complementation molecules can be assayed by comparing the target specific fluorescence of protein complementation molecules in the presence and absence of the target. The protein complementation regulator sequences present in these cells can be subsequently determined by amplification of these sequences using the PCR primers used to create the library and subsequent sequencing of the PCR products.

Thus, in some embodiments, methods of screening for protein complementation regulators are provided comprising the following steps: (a) introducing (i) a library of protein complementation regulators, (ii) an inducible target, (iii) a first complementation molecule comprising a first probe portion which binds to the induced target with requisite specificity and a first effector portion (e.g., comprising a first split fluorescent protein or a first ligand binding thereto), and (iv) a second complementation molecule comprising a second probe portion which binds to the induced target with requisite specificity and a second effector portion (e.g., comprising a second split fluorescent protein or a second ligand binding thereto) into cells; (b) (i) determining fluorescence of cells by FACS or fluorescence microscopy in uninduced cells and (ii) removing cells with high fluorescence (e.g., higher level than a pre-set value or higher level than background cell fluorescence in the absence of one or more of the molecules in step (a)) and/or selecting cells with low fluorescence (e.g., lower level than a pre-set value or the same or similar level as background cell fluorescence in the absence of one or more of the molecules in step (a)) as potential complementation regulators; and (c) (i) inducing cells to express the target, (ii) determining fluorescence of cells by FACS or fluorescence microscopy in induced cells, (iii) selecting cells with high fluorescence as potential protein complementation regulators (e.g., higher level than a pre-set value or a similar or higher level than the fluorescence in the presence of the induced target and first and second complementation molecules) and/or discarding cells with low fluorescence (e.g., lower level than a pre-set value or a similar or lower level than the fluorescence in the presence of the induced target and first and second complementation molecules). In some embodiments, step (a) is performed prior to step (b). In some embodiments, step (b) is performed prior to step (a). In some embodiments, the method further comprises creating a library of potential protein complementation regulator. In some embodiments, the potential protein complementation regulator is cloned between two known primer sequences. In some embodiments, the method further comprises amplifying the potential complementation regulator sequence using the primers sequences flanking same. In some embodiments, the method further comprises sequencing the potential protein complementation regulator sequence. In some embodiments, the method further comprises assaying the potential protein complementation regulator in the presence or absence of target and comparing to other protein complementation regulators and/or the complementation molecules in the absence of a protein complementation regulator. Also provided herein are protein complementation regulators identified by the method of screening for protein complementation regulators.

### Complementation Molecules

Provided herein are complementation molecules for use with the protein complementation regulators described herein. The complementation molecules comprise a probe portion and an effector portion. The probe portions of the complementation molecule are responsible for binding to the target, while the effector portions upon binding of the probe portions to the target are brought into proximity and interact with each other such as to form an assembled complex, directly, wherein the effector portion is the effector molecule or indirectly, for example, by recruiting an effector molecule to form an assembled complex.

In some embodiments, the probe portion is a nucleic acid and the effector portion is a polypeptide. In some embodiments, the probe protein is a polypeptide and the effector portion is a nucleic acid. In some embodiments, both the probe portion and the effector portion are polypeptides. In some embodiments, both the probe portion and the effector portion are nucleic acids.

The complementation molecule can be a single type of molecule (such as a complementation molecule comprising a probe portion and an effector portion) or be a chimeric biomolecular molecule comprising a probe portion associated to an effector portion.

The complementation molecule, in some embodiments, may be a conjugate comprising a probe portion conjugated to an effector portion. In some embodiments, the complementation molecule comprises a probe portion covalently linked to an effector portion. For covalent linkages, various functionalities can be used, such as amide groups, including carbonic acid derivatives, ethers, esters, including organic and inorganic esters, amino, urethane, urea and the like. To provide for linking, the effector portion and/or the probe portion can be modified by oxidation, hydroxylation, substitution, reduction etc. to provide a site for coupling. It will be appreciated that modification which do not significantly decrease the function of the effector portion and/or the probe portion are preferred.

In some embodiments, several linkers may be included in order to take advantage of desired properties of each linker and each molecule in the conjugate. For example, in some embodiments, the complementation molecule further comprises a linker region or linker domain (e.g., nucleic acid or polypeptide) between the first and second portions. The effector portion in a complementation molecule can be, for example, a ligand (such as an aptamer) capable of recruiting an effector molecule thereof to form an assembled complementation complex. Flexible linkers and linkers that increase the solubility of the conjugates are contemplated for use alone or with other linkers are incorporated herein. Linkers may be acid cleavable, photocleavable and heat sensitive linkers. The joining can be fusion of the entities, for example fusion of polypeptides. Conjugation can also be performed by other means known in the art, for example but not limited to covalent, ionic, or hydrophobic interaction whereby the moieties of a molecule are held together and preserved in proximity.

In some embodiments, more than two complementation molecules are used in the methods described herein. In some embodiments, the complementation molecules are of the same type. For example, all complementation molecules are nucleic acids or all complementation molecules are proteins. In some embodiments, the complementation molecules are of a different type. For example, one complementation molecule is a nucleic acid and another complementation molecule is a polypeptide. As another example, one complementation molecule can be a conjugate of two polypeptides and the other complementation molecule is a conjugate of a nucleic acid and a polypeptide.

The methods of the present application make use of complementation molecules. The probe portion on the complementation molecules bind to a target, thereby bringing the effector portions into proximity. The probe portions can be designed so that their binding to the target allows effective interaction between the effector portions and formation of an assembled complementation complex.

In some embodiments, when the complementation molecule is a nucleic acid or a polypeptide and there is a nucleic acid or peptide sequence between the probe portion and the effector portion, the length and sequence of the nucleic acid or peptide sequence between the two regions can be adjusted to optimize the interaction of the effector portions and formation of an assembled complementation complex. In some embodiments when a linker is used between the probe portion and the effector portion in the complementation molecule, the nature, length, and flexibility of the linker can be adjusted in order to optimize the interaction between the effector portions and formation of an assembled complementation complex.

Two or more parameters discussed above can also be adjusted simultaneously in order to optimize the interaction between the effector portions and formation of an assembled complementation complex. The flexibility of the design and ability to adjust multiple parameters makes the methods of the present application particularly advantageous over other methods, such as methods based on protein-protein interactions.

The probe portions described herein bind to a target with requisite specificity. In some embodiments, only one of the probe portion specifically recognizes (for example specifically hybridizes to) the target. In some embodiments, both probe portions specifically recognize (for example specifically hybridize to) the target nucleic acid. In some embodiments, the probe portions of the complementation molecules specifically, collectively associate with the target molecule. In some embodiments, the probe portions preferential associate to a target compared to a non-target molecules. For example, when the target is a result of the translocation of two different genes, one probe portion can recognize a sequence on one of the genes, while the other probe portion can recognize a sequence on the other gene. Although each probe portion can simultaneously bind to both a wild-type gene and a mutant gene resulting from translocation, the probe portions only bind to the mutant gene together and thus show specificity of binding.

The probe portions can be chosen so that they bind to regions of the target that is accessible to the probe portions. For example, when the target is an RNA, the probe portions can be chosen so that they bind to regions on the RNA are exposed and accessible by the probe portions. In some embodiments when the target nucleic acid is DNA, the probe portions can be chosen so that they bind to regions on the DNA that are exposed and accessible by the probe portions. In some embodiments, when the target is a polypeptide, the probe portions can be chosen so that they bind to regions which are in proximity based on the tertiary structure of the polypeptide and exposed and accessible by the probe portions.

The protein complementation regulator may be provided in cis or in trans with the complementation molecules. In some embodiments, the protein complementation regulator is associated with one or more of the complementation molecules. In some embodiment, the protein complementation regulator is coupled with one or more of the complementation molecules. In some embodiments, the protein complementation regulator is conjugated to one or more of the complementation molecules. In some embodiments, the protein complementation regulator is covalently linked to the complementation molecules. In some embodiments, the protein complementation regulator is covalently linked in cis to the complementation molecule and is 5′ or N-terminal to the first probe portion, between the first and second probe portion, between the first and second effector portion, or 3′ or C-terminal to the second probe portion. In some embodiments, the protein complementation regulator is covalently linked in cis to the complementation molecule and is between the first effector portion and the second effector portion. In some embodiments, the first and second effector portions are two fragments of a split aptamer capable of binding an effector molecule and the protein complementation regulator is covalently linked in cis to the complementation molecule and is between the first effector portion and the second effector portion.

### Probe Portions

The probe portions in the complementation molecules described herein provide binding to the target with requisite specificity. In some embodiments, the probe portion can be any molecule that is capable of binding to a target with requisite specificity. In some embodiments, the probe portion is a nucleic acid or a polypeptide. In some embodiments, the probe portion is a nucleic acid binding motif. In some embodiments, the nucleic acid binding motif is a nucleic acid sequence (e.g., oligonucleotide) or a polypeptide. In some embodiments, the probe portion is a polypeptide binding motif. In some embodiments, the probe portions are capable of binding to a target polypeptide which contains post-translational modifications with requisite specificity. In some embodiments, the probe portion preferentially binds to a post-translationally modified polypeptide. In some embodiments, the probe portions are capable of binding to a target organic molecule with requisite specificity. In some embodiments, the probe portions are molecules capable of interacting with a target inorganic molecule with requisite specificity.

In some embodiments, the probe portion is a nucleic acid. Examples of nucleic acid sequences useful as nucleic acid binding motifs include, but are not limited to, oligonucleotides; single stranded RNA molecules; and peptide nucleic acids (PNAs), pseudocomplementary PNA (pcPNA), locked nucleic acids (LNA), morpholin DNAs, phosphorthioate DNAs, and 2′-O-methoxymethyl-RNAs. In some embodiments, a nucleic acid binding motif is an oligonucleotide. Methods for designing and synthesizing oligonucleotides are well known in the art. Oligonucleotides are sometimes referred to as oligonucleotide primers. An oligonucleotide can hybridize to a polynucleotide template and act as a point of initiation for the synthesis of a primer extension product that is complementary to the template strand.

An oligonucleotide that can hybridize to a polynucleotide template and act as a point of initiation for the synthesis of a primer extension product that is complementary to the template strand. Many of the oligonucleotides described herein are designed to be complementary to certain portions of other oligonucleotides or nucleic acids such that stable hybrids can be formed between them. The stability of these hybrids can be calculated using known methods such as those described in Lesnick and Freier, Biochemistry 34:10807-10815 (1995), McGraw et al., Biotechniques 8:674-678 (1990), and Rychlik et al., Nucleic Acids Res. 18:6409-6412 (1990).

Oligonucleotides useful in the methods as disclosed herein can be synthesized using established oligonucleotide synthesis methods, which are well known by persons of ordinary skill in the art. Such methods can range from standard enzymatic digestion followed by nucleotide fragment isolation (see for example, Sambrook, et al., Molecular Cloning: A Laboratory Manual, Second Edition, Cold Spring Harbor, N.Y., (1989), Wu et al, Methods in Gene Biotechnology (CRC Press, New York, N.Y., 1997), and Recombinant Gene Expression Protocols, in Methods in Molecular Biology, Vol. 62, (Tuan, ed., Humana Press, Totowa, N.J., 1997), the disclosures of which are hereby incorporated by reference), to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol. 65:610-620 (1980), (phosphotriester method).

In some embodiments, the probe portion is a nucleic acid binding motif. In some embodiments, the nucleic acid binding motif is a nucleic acid binding moiety such as peptide nucleic acids (PNAs), including pseudocomplementary PNAs (pcPNA). Methods for designing and synthesizing PNAs and pcPNAs are well known by persons of ordinary skill in the art. Peptide nucleic acids (PNAs) are analogs of DNA in which the backbone is a pseudopeptide rather than a phosphodiester. Thus, their behavior mimics that of DNA and binds complementary nucleic acid strands. In peptide nucleic acids, the deoxyribose phosphate backbone of oligonucleotides has been replaced with a backbone more akin to a peptide than a sugar phosphodiester. Each subunit has a naturally occurring or non naturally occurring base attached to this backbone, for example a backbone can be constructed of repeating units of N-(2-aminoethyl)glycine linked through amide bonds.

PNA binds to both DNA and RNA to form a PNA/DNA or PNA/RNA duplex which bindswith greater affinity and increased specificity than corresponding DNA/DNA or DNA/RNA duplexes. In addition, the polyamide backbone of PNA (having appropriate nucleobases or other side chain groups attached thereto) is not recognized by either nucleases or proteases, and thus PNAs are resistant to degradation by enzymes, unlike DNA and peptides. The binding of a PNA strand to a DNA or RNA strand can occur in either a parallel of anti-parallel orientation. PNAs bind to both single stranded DNA and double stranded DNA.

In some embodiments, pseudocomplementary PNAs (pcPNAs) can be used which are a variation of PNA molecules and, in addition to guanine and cytosine, pcPNA's carry 2,6-diaminopurine (D) and 2-thiouracil instead of adenine and thymine, respectively. pcPNAs exhibit a distinct binding mode, double-duplex invasion, which is based on the Watson-Crick recognition principle supplemented by the notion of pseudocomplentarity. pcPNAs recognize and bind with their natural A, T, (U), or G, C counterparts. pcPNAs can be made according to any method known in the art. For example, methods for the chemical assembly of PNAs are well known (See U.S. Pat. No. 5,539,082, 5,527,675, 5,623,049, 5,714,331, 5,736,336, 5,773,571 or 5,786,571 which are incorporated herein by reference).

In some embodiments, one or more of the nucleic acid-based nucleic acid binding motifs are single stranded nucleic acids. In some embodiments, the single stranded nucleic acid-based nucleic acid binding motif forms a duplex upon binding to the target nucleic acid. In some embodiments, one or more of the nucleic acid-based nucleic acid binding motifs are double stranded nucleic acids. In some embodiments, nucleic acid binding motif is capable of binding to a target nucleic acid by triple helix formation.

The nucleic acid binding motif can also be a polypeptide having a high binding affinity for the target nucleic acid, such as a peptide (for example peptides of less than about 100 amino acids), a full length protein, or a fragment of a protein (such as a nucleic acid binding domain of a protein). For example, a polypeptide nucleic acid binding motif can contain multiple domains (for example zinc finger motifs) or a nucleic acid binding molecule which has been split into two separate components, such as eIF-4A. In some embodiments, the polypeptide nucleic acid binding motif's affinity for the target nucleic acid is in the low nanomolar to high picomolar range. Polypeptides useful in the methods as disclosed herein include polypeptides which contain zinc fingers, either natural or designed by rational or screening approaches. Examples of zinc fingers include Zif 2g8, Sp1, finger 5 of Gfi-1, finger 3 of YY1, finger 4 and 6 of CF2II, and finger 2 of TTK (PNAS 97:1495-1500 (2000); J Biol Chem 276 (21):29466-78 (2001); Nucl Acids Res 29(24):4920-9 (2001); Nucl Acid Res 29(11):2427-36 (2001)).

Other polypeptides which are useful in the methods as disclosed herein include polypeptides, obtained by in vitro selection, that bind to specific nucleic acids sequences, for example, peptide aptamers such as aptamers of platelet-derived growth factor (PDGF) (Nat Biotech 20:473-77 (2002)) and thrombin (Nature 355:564-6 (1992)). Other polypeptides useful in the methods as disclosed herein include polypeptides which bind to DNA triplexes in vitro; for example, members of the heteronuclear ribonucleic particles (hnRNP) proteins such as hnRNP K, L, E1, A2/B1 and I (Nucl Acids Res 29(11):2427-36 (2001)).

In some embodiments, the nucleic acid-based nucleic acid binding motifs can bind to the same hybridization site on a single-stranded target, creating a triplex at the hybridization site comprising the target nucleic acid and nucleic acid binding motifs hybridizing the same site. The nucleic acid binding motif can be any nucleic acid which allows triplex formation. In some embodiments, triplex-forming oligonucleotides are GC-rich, for example a purine triplex, consisting of pyrimidine-purine-purine.

In some embodiments, the probe portion is a protein binding motif. Examples of protein binding motifs are known in the art. See e.g., U.S. Pat. No. 6,270,964, U.S. Pat. No. 6,294,330, and/or U.S. Pat. No. 6,897,017, which are incorporated by reference in their entirety. In some embodiments, the protein binding motif recognizes one or more components of a multimeric complex. In some embodiments, the multimeric complex is a dimer. In some embodiments, the dimer is a homodimer or heterodimer. In some embodiments, the dimer is an ErbB2/ErbB3 heterodimer. For examples of multimeric complex targets are described in Holbro et al, PNAS 100(15):8933-8 (2003), which is incorporated by reference in its entirety.

In some embodiments, the probe portions of the complementation molecules can be the same kind of molecule, for example both probe portions can be oligonucleotides, or they can be different, for example, one probe portion of a complementation molecule can be an oligonucleotide and the probe portion of another complementation molecule can be a polypeptide.

For example, the probe portions can be designed to specifically bind to two adjacent regions of a target. In some embodiments, the two adjacent regions may be adjacent along target sequence (e.g., a nucleic acid strand and/or along a multiplex (e.g., duplexed) nucleic acid strand). In some embodiments, the two adjacent regions are adjacent in space, but not necessarily adjacent to each other on the target sequence (e.g., same nucleic acid strand and/or along a multiplex (e.g., duplexed) nucleic acid strand). The distance between the two target regions can be adjusted (by choosing different probe portions) to allow optimized interaction between the effector portion and/or the effector molecules recruited by the effector portions. In some embodiments, the two target regions overlap with each other. In some embodiments, the two regions are separated by no more than about 20, 40, 60, 80, 100, 200, or 300 nucleotides. In some embodiments, the two regions are separated by at least about 20, 40, 60, 80, 100, 200, or 300 nucleotides.

Alternatively, nucleic acid-based nucleic acid binding motifs can bind to closely adjacent hybridization sites on a single-stranded or double-stranded target nucleic acid, creating either a duplex or a triplex at each hybridization site, respectively. In some embodiments, the nucleic acid binding motifs may bind to sites greater than about any of 5, 10, 15, 20, 25, 30, 35, 40, 45, 50, 75, 100, 250, or 500 nucleotides apart. In some embodiments, the nucleic acid binding motifs may bind to sites less than about any of 5, 10, 15, 20, 25, 30, 35, 40, 45, 50, 75, 100, 250, or 500 nucleotides apart. In some embodiments, the nucleic acid binding motifs may bind to sites about any of 5, 10, 15, 20, 25, 30, 35, 40, 45, 50, 75, 100, 250, or 500 nucleotides apart.

The nucleic acid-based nucleic acid binding motifs can be complementary to the corresponding target sequence. In some embodiments, the nucleic acid binding motifs are specifically hybridizable to the corresponding target sequence. In some embodiments, the nucleic acid binding motifs are at least about 80% (including for example at least about any of 90%, 95%, 99%) identical to the corresponding sequence on the target nucleic acid. Each of the nucleic acid binding motifs can range from about any of 5-30, 5-15, 5-20, 5-21, 5-22, 5-23, 5-24, 5-30, 5-35, 5-40, or 5-50 nucleotides long. In some embodiments, the nucleic acid binding motif may be about any of 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, the nucleic acid binding motif may be greater than about any of 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, the nucleic acid binding motif may be less than about any of 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, the nucleic acid binding motif may be greater than about any of 5, 6, 7, 8, 9, 10, 11, 12, 13, 14, 15, 16, 17, 18, 19, 20, 21, 22, 23, 24, 25, 26, 27, 28, or 29 nucleotides and less than about any of 6, 7, 8, 9, 10, 11, 12, 13, 14, 15, 16, 17, 18, 19, 20, 21, 22, 23, 24, 25, 26, 27, 28, 29, or 30 nucleotides. In some embodiments, the nucleic acid binding motif can be more than 30 nucleotides long.

The length, sequence, and the combined length of the nucleic acid binding motifs can be adjusted in order to obtain a desired binding specificity and stability of the complex. For example, in some embodiments when two complementation molecules are used, the combined lengths of the first and second nucleic acid binding motifs can be less than about any of 20, 30, 40, 50, 60, 80, or 100 nucleotides. In some embodiments, the combined lengths of the first and second nucleic acid binding motifs is more than about any of 20, 30, 40, 50, 60, 80, or 100 nucleotides. The first and second nucleic acid binding motifs can be of the same or different lengths.

In some embodiments, the nucleic acids encompasses nucleic acids containing analogs and derivatives of natural nucleotides, which have similar binding properties as the reference nucleic acid and may or may not be metabolized in a manner similar to naturally occurring nucleotides. In some embodiments, a particular nucleic acid sequence, as described herein, may also encompasses conservatively modified variants thereof (e.g., degenerate codon substitutions) and complementary sequences. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985), and Rossolini et al., Mol. Cell. Nucleic acid binding motifs 8:91-98 (1994)).

### Effector Portions

The effector portions in the complementation molecules described herein are responsible for assembly of a functional complementation complex which has a desired functional effect. A desired functional effect includes but is not limited to generating a detectable signal (e.g., fluorescent protein), inducing cell death, inducing a cytotoxic effect, sensitizing cells, degrading nucleic acids or polypeptides, promoting cell survival or replacing a lost and/or dysfunctional polypeptide etc.

In some embodiments, the effector portion is an effector molecule. In some embodiments, the effector molecule is a polypeptide fragment. In some embodiments, the effector molecule is a functional polypeptide fragment. In some embodiments, the effector molecule is a polypeptide.

In some embodiments, the effector portion is a ligand that binds with high affinity to an effector molecule and recruits an effector molecule to the target, thereby forming an assembled complementation complex. A ligand can be any molecule that binds to the effector molecule with high affinity. The binding of the ligand to the effector molecule should be strong enough to recruit the effector molecule. For example, in some embodiments, the ligand binds to the effector molecule with an affinity of about any of 10 nM, 50 nM, 100 nM, 500 nM, 1 μM, 5 μM, or 10 μM.

In some embodiments, the effector molecule is a nucleic acid. In some embodiments, the effector molecule is a polypeptide. In some embodiments, the ligand for the effector molecule is an antibody. In some embodiments, the ligand for the effector molecule is a non-antibody polypeptide.

In some embodiments, the ligand for the effector molecule is an aptamer that binds to the effector molecule with high affinity and specificity. In some embodiments, the aptamer is about any of 5, 6, 7, 8, 9, 10, 11, 12, 13, 14, 15, 16, 17, 18, 19, or 20 kDa. In some embodiments, the aptamer binds to the effector molecule or fragment thereof by one or more of hydrogen bonding, electrostatic complementarity, hydrophobic contacts, or steric exclusion.

In some embodiments, the aptamer is a nucleic acid aptamer. In some embodiments, the nucleic acid aptamer is an RNA aptamer. In some embodiments, the nucleic acid aptamer is a DNA aptamer. In some embodiments, the DNA aptamer is a single stranded DNA. In some embodiments, the DNA aptamer is double stranded DNA. In some embodiments, the nucleic acid aptamer has defined tertiary structures for binding. In some embodiments, the nucleic acid aptamer is between about any of 5-10, 10-15, or 15-20 kDa in size and/or about any of 20-30, 30-40, 40-50, 50-60, or 60-80 nucleotides long.

An aptamer that specifically binds to a target nucleic acid or target polypeptide can be made by the SELEX (systematic evolution of ligands by exponential enrichment) process described in U.S. Pat. Nos. 5,475,096 and 5,270,163 (incorporated herein by reference). Briefly, a commonly used procedure of SELEX involves immobilizing a selected target on a column. A solution containing a library or assortment of random DNA or RNA molecules or polypeptides is contacted with the target under conditions that are favorable for binding. After a certain amount of time the column is flushed and those aptamer molecules which bind to the target remain on the column. The unbound molecules will be eluted from the column. The aptamer-target complexes are then dissociated and the aptamer molecules that were selected by binding to the target are analyzed for example by amplification. The cycle may be repeated to achieve a higher affinity aptamer.

In some embodiments, the aptamer is a smart aptamer, wherein the aptamer is selected with pre-defined equilibrium dissociation constant (Kd), dissociation and association rate constants (k_{off} and kₒₙ, respectively), and/or thermodynamic (ΔH, ΔS) parameters of aptamer-target interaction.

In some embodiments, the aptamer is modified by, for example, 2′-fluorine-substituted pyrimidines, polyethylene glycol (PEG) linkage, etc. In some embodiments, the modification of the aptamer increases the serum half-life of aptamers by about any of days or weeks.

In some embodiments, the aptamer is a peptide aptamer. The peptide aptamer generally consists of a variable peptide loop attached at both ends to a protein scaffold. In some embodiments, the variable loop length of the aptamer is about any of 5, 6, 7, 8, 9, 10, 11, 12, 13, 14, 15, 16, 17, 18, 19, 20, 21, 22, 23, 24, or 25 amino acids. In some embodiments, the variable loop length of the aptamer is between about any of 10 to 20 amino acids. In some embodiments, the scaffold may be any protein which has good solubility and capacity properties. In some embodiments, the scaffold protein is Thioredoxin-A. In some embodiments, the variable loop is inserted within the reducing active site, which is a -Cys-Gly-Pro-Cys-loop in the Thioredoxin-A, the two Cysteines lateral chains being able to form a disulfide bridge.

In some embodiments, the aptamer is a Ligand Regulated Peptide Aptamer (LiRPA).

In some embodiments, the peptide aptamer selection can be made using the yeast two-hybrid system. In some embodiments, the aptamer is selected is made by AptaBiD (Aptamer-Facilitated Biomarker Discovery). AptaBiD is based on multi-round generation of an aptamer or a pool of aptamers for differential molecular targets.

Exemplar aptamers and methods of making aptamers are described in Wilson and Szostak, Annu. Rev. Biochem. 68:611-647 (1999), Mayer, Angew. Chem. Int. Ed. 48:2672-2689 (2009), and Bouchard et al., Annu. Rev. Pharmacol. Toxicol 50:237-57 (2010), which are incorporated herein by reference in their entirety.

### Effector Molecules

Various effector molecules are provided herein. In some embodiments, an effector molecule is a fluorescent protein, for example, but not limited to, green fluorescent protein (GFP), enhanced green fluorescent protein (EGFP), green-fluorescent-like proteins; yellow fluorescent protein (YFP), enhanced yellow fluorescent protein (EYFP), blue fluorescent protein (BFP), enhanced blue fluorescent protein (EBFP), cyan fluorescent protein (CFP), enhanced cyan fluorescent protein (ECFP), a red fluorescent protein (dsRED), or derivatives thereof, where one of the fragments in the reconstituted fluorescent protein contains a mature preformed chromophore. All of the above mentioned fluorescent proteins and fragments thereof that will result in a fluorescing fluorescent protein are encompassed within the methods described herein. Also encompassed are those fluorescent proteins known to those of skill in the art, and fragments and genetically engineered proteins thereof.

In some embodiments, the presence of an active effector protein, for example an active fluorescent protein is detectable by flow cytometry, fluorescence plate reader, fluorometer, microscopy, fluorescence resonance energy transfer (FRET), by the naked eye or by other methods known to persons skilled in the art. In some embodiments, fluorescence is detected by flow cytometry using a florescence activated cell sorter (FACS) or time lapse microscopy.

In some embodiments, an effector molecule is an enzyme, such that the effector molecules(s) or fragments thereof associate in close proximity to form an assembled, active enzyme, which can be detected using an enzyme activity assay. Preferably, the enzyme activity is detected by a chromogenic or fluorogenic reaction. In one preferred embodiment, the enzyme is dihydrofolate reductase (DHFR) or β-lactamase.

In some embodiments, the enzyme is dihydrofolate reductase (DHFR). For example, Michnick et al. have developed a “protein complementation assay” consisting of N- and C-terminal fragments of DHFR, which lack any enzymatic activity alone, but form a functional enzyme when brought into close proximity. See e.g. U.S. Pat. Nos. 6,428,951, 6,294,330, and 6,270,964, which are hereby incorporated by reference. Methods to detect DHFR activity, including chromogenic and fluorogenic methods, are well known in the art.

In some embodiments, other effector molecules can be used, for example, enzymes that catalyze the conversion of a substrate to a detectable product. Several such enzymes include, but are not limited to β-galactosidase (Rossi et al., PNAS 94; 8405-8410 (1997)); dihydrofolate reductase (DHFR) (Pelletier et al., PNAS 95; 12141-12146 (1998)); TEM-I β-lactamase (LAC) (Galarneau et al., Nat Biotech 20; 619-622 (2002)) and firefly luciferase (Ray et al., PNAS 99; 3105-3110 (2002)) and Paulmurugan et al., PNAS 99; 15608-15613 (2002)). For example, split β-lactamase has been used for the detection of double stranded DNA (see Ooi et al., Biochemistry; 45; 3620-3525 (2006)).

In some embodiments, the effector molecule(s) are fragments which are preformed and/or fully folded into a mature conformation and therefore useful in rapid complementation, for example rapid signal detection. In some embodiments, the effector molecule(s) which are preformed and/or fully folded is useful in real-time signal detection. In some embodiments, the effector molecule which are preformed and/or fully folded are polypeptides. In some embodiments, the polypeptide is a chromophore (e.g., CFP, GFP, EGFP, YFP, or RFP)

Cytotoxic Effector Molecules

In some embodiments, an effector molecule is a cytotoxin, for example a bacterial toxin, bacterial cytotoxin, plant-derived toxin, or plant-derived cytotoxin. Cytotoxins are well known to persons skilled in the art for example but not limited to anthrax toxin; diphtheria toxin (DT); ricin A toxin (RTA); pseudomonal endotoxin (PE); streptolysin O; saporin; gelonin or naturally occurring variants, or genetically engineered variants or fragments thereof. Bacterial toxins are typically not glycosylated, but glycosylated bacterial toxins are also encompassed within the methods described herein. For example, DT has been genetically modified to improve their specificity and non-specific binding to normal cells, for example DT is mutated by converting leu 390 and Ser525 each to phenylalanine, resulting in CRM107 (Greenfield et al., Science 238:536-539 (1987)), or DT and PE, including PE40, have been truncated. For variations of mutations and modifications to modify the properties of bacterial toxins used, see the review Kreitman R. J, APPS Journal 8(3); E532-E551 (2006), incorporated in its entirety herein for reference.

In some embodiments, the effector molecule is a plant toxin. Plant toxins are well known to persons skilled in the art and can be a plant halotoxin or class II ribosome inactivating protein, or a hemitoxin or class I ribosome inactivating protein. A plant halotoxin can be for example, but not limited to saporin (SAP); pokeweed antiviral protein (PAP); bryodin 1; bouganin and gelonin or naturally occurring variants, or genetically engineered variants or fragments thereof. A plant hemitoxin can be, for example ricin A chain (RTA); ricin B (RTB); abrin; mistletoe, modeccin or naturally occurring variants, or genetically engineered variants or fragments thereof. Plant toxins are typically glycosylated, but non-glycosylated plant toxins are also encompassed within the methods described herein. In some embodiments, the plant toxins can function as nucleases, for example, but not limited to sarcin; restrictocin.

In some embodiments, the effector molecule comprises a polypeptide or fragment of a cytotoxic molecule or protein. One example of a cytotoxic molecule is cytokine. Non-limiting examples of cytokines that have been used as toxins for cancer include IL-1; IL-2 (CD25); IL-3; IL-4; IL-13; interferon-alpha; tumor necrosis factor-alpha (TNFα); IL-6; granulocyte-macrophage colony stimulating factor (GM-CSF); G-CSF. The cytokines can be or natural occurring variants of cytokines or alternatively been genetically engineered variants thereof, or cytokines comprising a heterologous sequence of recombinant cytokines.

In some embodiments, the effector molecule is a humanized immunotoxin that comprises a human or mammalian toxin, for example but not limited to RNase, protamine/DNA, and Bax. For review of examples of humanized toxins, see review by Frankel, A., Clinical Cancer Res 10:13-15 (2004), which is incorporated herein in its entirety by reference.

Nucleases Effector Molecules

In some embodiments, an effector molecule is a nuclease or has endonucleolytic activity. In some embodiment the nuclease is a DNA nuclease, DNA endonuclease, or DNA exonuclease. The nuclease can be a natural variant, homologue or a genetically modified variant thereof. Examples of known DNA endonucleases are well known to persons skilled in the art, and have been used for conjugates for immunotoxins (see WO01/74905, which is incorporated herein in its entirety by reference, and include examples, such as bovine DNase I (see Worrall and Conolly, J. Biol. Chem. 265; 21889-21895 (1990)); pancreatic DNase I (see Shak et al., Proc. Natl. Acad. Sci. USA., 87; 9188-9192 (1990) and Hubbard et al., New Eng. J. Med. 326:812-815 (1992)). In some embodiments, the DNase nuclease is a mammalian deoxyribonuclease I, and in others it is a human deoxyribonuclease I.

In some embodiments, the nuclease is an RNA nuclease, RNA endonuclease or RNA exonuclease. RNA nucleases are well known to persons skilled in the art, any of which are encompassed within the methods described herein. Non-limiting examples of RNA nucleases include RNA endonuclease I; RNA endonuclease II; RNA endonuclease III. In some instances, the RNase can be a ribonuclease A (RNase A), one such example is referred to the trade name of Onconase®, (available from Tamir Biotechnology, Inc., Bloomingfield, N.J.) derived from Rana pipens oocytes that was originally designated P-30. One of skill will appreciate any RNase or RNase A molecule can be modified using numerous methods known to those skilled in the art, and use of such modified or recombinant RNase and/or RNase A molecules, or naturally occurring variants thereof, as effector molecules are encompassed for use in the methods as disclosed herein. In some embodiments, the RNase is a bacterial RNase. In some embodiments, the RNase is a fungal RNase. In some embodiments, the RNase is a eukaryotic RNase. In some embodiments, RNase A can be used as an effector molecule which has been disclosed in the use as an immunotoxin in European Patent Application EP975671; U.S. Pat. No. 6,869,604, which are incorporated herein by reference, which use ribonuclease derived from Rana pipiens. In other embodiments, ribonucleases derived from Rana catesbeiana oocytes can be used as effector molecules. Although the amino acid sequence of Rana catesbeiana oocyte RNase (RCOR1) has been known since 1989, genomic DNA or mRNA which encodes oocyte RNases and genetically modified variants thereof are also encompassed. In some embodiments, RNases useful in the methods as disclosed herein can be of the superfamily of human pancreatic RNases, for example human angiogenin or a fragment thereof, or a recombinant or genetically engineered variant thereof having ribonuclease activity (Kurachi et al., Biochemistry 24; 5494-5499 (1985)). Angiogenin is also a potent inhibitor of protein synthesis in cell-free extracts and upon injection into Xenopus oocytes. Extracellular angiogenin is not cytotoxic towards a wide variety of culture cells and is normally present in human plasma therefore its reconstitution within a cell is an ideal candidate as an effector molecule in the split-biomolecular conjugate. Further, human angliogenin has been used in immunotherapy by conjugating to IL2, see European Patent Application EP 1217070, incorporated herein in its entirety for reference, and has also been shown to be expressed as two portions of two human proteins or fragments thereof.

In some embodiments, an RNase can be Dicer. Dicer or Dcr-1 homolog (Drosophila) is an RNAse III nuclease that cleaves double-stranded RNA (dsRNA) and pre-microRNA (miRNA) into short double-stranded RNA fragments of about 20-25 nucleotides long, usually with a two-base overhang on the 3′ ends (often called small interfering RNA (siRNA)). Because dicer contains two RNase domains and one PAZ domain; an effector molecule could comprise each domain of Dicer. Dicer catalyzes the first step in the RNA interference pathway and initiates formation of the RNA-induced silencing complex (RISC), whose catalytic component argonaute is an endonuclease capable of degrading messenger RNA (mRNA) whose sequence is complementary to that of the siRNA guide strand.

In some embodiments, the nuclease is a restriction endonuclease, for example microbial type II restriction endonucleases. Exemplary but non-limiting examples of type II restriction endonucleases include; BamHI; Hind III; Msp1; Sau3AI; Hinf1; Not1; and EcoRI.

Other Effector Molecules

In some embodiments, an effector molecule is a proteolytic enzyme which break peptide bonds between amino acids of proteins by a process called proteolytic cleavage and is a common mechanism of activation or inactivation of enzymes. Some proteases use a molecule of water for proteolytic cleavage and are also classified as hydrolases. Proteases useful in the methods as discloses herein are well known by persons skilled in the art, and include for example, but are not limited to, serine proteases; threonine proteases; cysteine proteases; aspartic acid proteases (e.g., plasmepsin); metalloproteases; glutamic acid proteases; endopeptidases (proteinases) and exopeptidases. Common proteases are, for example; caspase enzymes; calpain enzymes; cathepsin enzymes; endoprotease enzymes; granzymes; matrix metalloproteases; pepsins; pronases; proteases; proteinases; rennin; trypsin, and their use, or naturally occurring homologues or genetically engineered variants thereof are encompassed within the methods described herein.

In some embodiments, an effector molecule is any molecule capable of inducing a cell death pathway in a cell. Examples of such effector molecules include, but are not limited to, pro-apoptotic molecule which are well known in the art, for example but not limited to Hsp90; TNFα; DIABLO; BAX; BID; BID; BIM; inhibitors of Bcl-2; Bad; poly ADP ribose polymerase-1 (PARP-I); Second Mitochondria-derived Activator of Caspases (SMAC); apoptosis inducing factor (AIF); Fas (also known as Apo-1 or CD95); Fas ligand (Fas L) are encompassed for use as effector molecules by the methods as disclosed herein, as well as natural variants or recombinant or genetically modified variants of such pro-apoptotic molecules.

In some embodiments, an effector molecule is capable of inhibiting a cell death pathway or inducing a cell survival pathway in the cell. Examples of such molecules include, but are not limited to numerous anti-apoptotic molecules which are well known by person of ordinary skill in the art, for example but not limited to; Bcl-2; BcI-XL; Hsp27; inhibitors of apoptosis (IAP) proteins.

In some embodiments, an effector molecule is a molecule or polypeptide that sensitizes the cell to one or more secondary agents. For example, an effector molecule can be a tyrosine kinase, for example β glucuronidase activity, β-Glucuronidase activates the low-toxic prodrugs such as 9-aminocamptothecin and p-hydroxy aniline mustard, or analogue such as a N-[4-doxorubicin-N-carbonyl(oxymethyl) phenyl] O-β-glucuronyl carbamate (DOX-GA3) have been developed to improve the antitumor effects of doxorubicin (DOX). The prodrug DOX-GA3 was initially designed to be activated into an active molecule or drug by human β-glucuronidase (GUS) to result in a highly cytotoxic effect specifically in the tumor site. The potency of such prodrugs can also be greatly enhanced with the incorporation of an appropriate radionuclide in a combined chemo- and radio-therapy of anti-cancer (CCRTC) strategy. In some embodiments, the prodrug can also be utilized to modify liposomes for efficient delivery of anti-cancer drugs (Chen et al., Current Medicinal Chemistry 3; 139-150 (2003)).

In some embodiments, an effector molecule that sensitizes the cell to another agent is, for example, hypoxanthine-guanine phosphoribosyltransferase (HGPRT), from the parasite Trypanosoma brucei (Tb), which can convert allopurinol, a purine analogue, to corresponding nucleotides with greater efficiency than its human homologue, therefore is capable of activating the prodrug allopurinol to a cytotoxic metabolite (Trudeau et al., Human Gene Ther 12:1673-1680 (2001)). In some embodiments, the effector molecule can be the bacterial nitrobenzene nitroreductase (NbzA) from Pseudomonas pseudoalcaligenes JS45, which activates the dinitrobenzamide cancer prodrug CB 1954 and the proantibiotic nitrofurazone (Berne et al., Biomacromolecules, 7; 2631-6 (2006)).

In some embodiments, an effector molecule is β-lactamase, which produces active agents or drugs from the pro-drug desacetylvinblastine-3-carboxylic acid hydrazide (DAVLBHYD) or other analogues. In such an embodiment, the Enterobacter cloacae beta-lactamase (bL) as an effector protein can activate the anticancer prodrugs 7-(4-carboxybutanamido)cephalosporin mustard (CCM), a cephalosporin prodrug of phenyl enediamine mustard (PDM) (Svensson et al., J Med. Chem. 41:1507-12 (1999)). Other prodrug/enzyme combinations known in the art can be used as the effector molecule and are encompassed for use in the methods as disclosed herein, including enzymes that produce toxic radicals on photodynamic therapy, for example peroxidase genes can be used as effector molecules.

In some embodiments, the effector molecule is a thymidine kinase. In some embodiments, the thymidine kinase is used for pro-drug therapy with thymidine analogs including, but not limited to, ganciclovir or AZT.

In some embodiments, an effector molecule is a molecule that catalyzes an antiviral drug, for example, but not limited to Oseltamivir which is commonly used as an anti-viral drug and can act as a secondary agent for carboxylesterase HCE1 as an effector molecule.

In some embodiments, an effector molecule can initiate addition or modification of a target nucleic acid or target polypeptide molecule. As a non-limiting example, where the target is a target polypeptide, a useful effector molecule can be ubiquitin, which adds, by covalent attachment, one or more ubiquitin monomers and tag the target polypeptide to be degraded via the proteasome. As another example where the target is a polypeptide, the effector molecule can be a Small Ubiquitin-related Modifier (SUMO) which tags the target polypeptide for numerous effects, including increased polypeptide stability, cellular localization etc. Other post-transcriptional events are known to persons skilled in the art, and include for instance; ISGylation; acetylation, alkylation, methylation biotinylation, glutamylation, glycylation; glycosylation, isoprenylation, lipoylation, phosphopantetheinylation, citrullination; deamidation, phosphorylation, etc., and the molecules that mediate or affect these events can be used as effector molecules.

In some embodiments, where the target is a target nucleic acid, an effector molecule useful in the methods as disclosed herein can modify the nucleic acid, for example chemical modification, includes, for example methylation or structural modification, for example acetylation or addition of histones to silence the gene and/or to prevent the transcription of the target nucleic acid. In one embodiment, an effector molecule can be a DNA methyltransferase (DNA MTase), for example, DNMT1, DNMT2, DNMT3A, DNMT3B or de novo methyltransferases or fragments thereof which will methylate the target nucleic acid on protein complementation. In another embodiment, an effector molecule is a histone acetyltransferase enzymes (HATs), such as CREB-binding protein, or modified version or variant thereof.

### Targets

The target which is specifically recognized by the complementation molecules can be one or more nucleic acid, one or more polypeptide, or one or more of any organic or inorganic molecule.

In some embodiments, the target is one or more nucleic acid molecule. The target nucleic acid can be DNA or RNA. The target nucleic acid can be a double-stranded, triple-stranded, or single-stranded DNA or RNA. In some embodiments, the target is a polypeptide. In some embodiments the target polypeptide is a polypeptide with specific posttranslational modifications.

A target sample includes, but is not limited, to target cells, target body fluids, and/or target lysates. Body fluids include but are not limited to blood, urine, cerebrospinal fluid, semen and tissue excudates. In some embodiments, the target is only present in the target sample i.e., the target is a target cell-specific molecule.

In some embodiments, the target is found at higher levels in a target sample compared to a non-target sample (e.g., higher levels in a target cell compared to the average levels in a non-target cell). In some embodiments, the target is present at greater than about any of 1.5 fold, 2 fold, 5 fold, 10 fold, 25 fold, 50 fold, or 100 fold more in a target sample compared to a non-target sample. In some embodiments, the target is present at about any of 1.5 fold, 2 fold, 5 fold, 10 fold, 25 fold, 50 fold, or 100 fold more in target sample compared to a non-target sample. The complementation molecules can be designed such that assembly of a functional complementation complex is formed only in a target sample with increased levels of the target. This can be achieved, for example, by adjusting the affinity of the probe portions and/or the amount of the complementation molecules introduced into the cells. Alternatively, effector portions can be chosen so that they only form an assembled complementation complex when the target is present at high concentrations and/or increased levels.

In some embodiments, the target nucleic acid is uniquely expressed in the target cell. In some embodiments, the target nucleic acid is not expressed in the non-target cell. For example, the target nucleic acid may be a variation of the corresponding nucleic acid in normal cells. The variation (mutation or polymorphism) may include, but is not limited to one or more of a deletion, a substitution, an insertion, a rearrangement, translocation, alternative slicing, single nucleotide polymorphism, etc. In some embodiments, a nucleic acid mutation or polymorphism will result in an altered polypeptide (e.g., missense mutation or nonsense mutation). In some embodiments, a nucleic acid mutation or polymorphism will not result in an altered polypeptide (e.g., silent mutation). This includes but is not limited to, for example, nucleic acid sequences encoding a mutation and/or polymorphism in a gene; regulatory sequence operatively linked to a gene or in the 5′ or 3′ untranslated regions (UTR) of a gene. For example, mutations and polymorphisms may contribute to the disease disorder or malignancy, or alternatively may contribute to the responsiveness of an individual or cell to a therapy with particular pharmaceutical agents (this is often termed “pharmacogenomics”). Similarly, mutations and/or polymorphisms can identify individuals or cells which may not function correctly due to expression of a dysfunctional protein which is toxic to the cell, thus identifies individual and cells that have increased likelihood of developing a disease, or the cells or individual being responsive or not responsive to a treatment. In some embodiments, pharmacogenomics can also be used in pharmaceutical research to assist the drug development and selection process. (See, e.g., Linder et al. (1997) Clinical Chemistry 43:254; Marshall (1997) Nature Biotechnology, 15, 1249; International Patent Application WO 97/40462, Spectra Biomedical; and Schafer et al. (1998) Nature Biotechnology 16:3).

In some embodiments, when the target is nucleic acid, the target nucleic acid is DNA. For example, the target nucleic acid can be part of the genomic DNA carrying a mutation that specifically identifies the target cell. In some embodiments, the target nucleic acid is a mutant gene resulting from a translocation event.

In some embodiments, the target nucleic acid is RNA. For example, the target nucleic acid can be an RNA biomarker for the target cell. In some embodiments, the target nucleic acid is an alternatively spliced form of an mRNA. In some embodiments, the target nucleic acid is an mRNA carrying a mutation that is characteristic to the target cell.

In some embodiments, the target is one or more target polypeptide. In some embodiments, the polypeptide is a multimeric complex. In some embodiments, the multimeric complex is a dimer. In some embodiments, the dimer is a homodimer or heterodimer. In some embodiments, the target polypeptide is a pathological polypeptide which contributes to part, or wholly, one or more symptoms of a disease, disorder or malignancy. In some embodiments, a pathological polypeptide is any protein that contributes to one or more symptoms of a disease due to dysfunctional or abnormal expression. For example, but not limited to, a pathogenic polypeptide can be a protein that is mutated, unfolded, in an abnormal conformation, in the incorrect subcellular location, expressed in an inappropriate cell and/or tissue types, inappropriately associated with another protein and/or lacking association with another protein. As an illustrative example only, a pathogenic polypeptide can be a protein that contributes to a symptom of a disease such as cancer, for example such pathogenic polypeptide can be pro-angiogenic proteins including, but not limited to, FGF, VEGF or PDGF, or contribute to neurodegenerative diseases such as β-amyloid in Alzheimer's disease; mutant SOD1 in amyotrophic lateral sclerosis (ALS) etc.

In some embodiments, a pathological polypeptide can be a polypeptide expressed on the surface of a pathogen, for example polypeptides that form part of the coat protein or capsid of virus particles, as a non-limiting example, the gp40 expressed on HIV virus particle, or other surface markers expressed on cancer cells, viruses or infectious particles.

In some embodiments, the target polypeptide is a polypeptide with specific posttranslational modifications. In some embodiments, the polypeptide is a polypeptide which displays aberrant posttranslational modifications which are different from posttranslational modifications found on the native protein in its natural cellular environment. Posttranslational modification include, but are not limited, to glycosylation, acetylation, phosphorylation, attachment of lipid moieties, attachment of other proteins (e.g., SUMOylation), and/or changing the chemical nature of amino acids (e.g. citrullination, formation of disulfide bonds, etc.)

The target cells described herein can be any cell, prokaryotic or eukaryotic, including plant, yeast, worm, insect and mammalian. Mammalian cells include, without limitation; primate cells, human cells and a cell from any animal of interest, including without limitation; mouse, hamster, rabbit, dog, cat, domestic animals, such as equine, bovine, murine, ovine, canine, feline and transgenic animals etc. The cells may be from a wide variety of tissue types without limitation such as; hematopoietic, neural, mesenchymal, cutaneous, mucosal, stromal, muscle spleen, reticuloendothelial, epithelial, endothelial, hepatic, kidney, pancreatic, gastrointestinal, pulmonary, T-cells, stem cells, embryonic stem (ES) cells, ES-derived cells and stem cell progenitors are also included, including without limitation, hematopoeitic, neural, stromal, muscle, cardiovascular, hepatic, pulmonary, gastrointestinal stem cells, etc. Yeast cells may also be used as cells. Cells also refer not to a particular individual cell but to the progeny or potential progeny of such a cell because of certain modifications or environmental influences, for example differentiation, such that the progeny may not, in fact be identical to the parent cell, but are still included.

In some embodiments, the target cell is a diseased cell (such as a cancer cell). In some embodiments, the target cell is a lymphocyte. In some embodiments, the target cell is a breast cell. In some embodiments, the target cell is an ovarian cell. In some embodiments, the target cell is a lung cell. In some embodiments, the target cell is a stem cell. In some embodiments, the target cell is a cancer stem cell. In some embodiments, the target cell is a pancreatic cell. In some embodiments, the pancreatic cell is an islet cell. In some embodiments, the target cell is a brain cell (such as neurons).

In some embodiments, the cancer cells are (I) the bone marrow and bone marrow derived cells (leukemias), (II) the endocrine and exocrine glands cells e.g., thyroid, parathyroid, pituitary, adrenal glands, salivary glands, pancreas, (III) the breast cells, like e.g., benign or malignant tumors in the mammary glands of either a male or a female, the mammary ducts, adenocarcinoma, medullary carcinoma, comedo carcinoma, Paget's disease of the nipple, inflammatory carcinoma of the young woman, (IV) the lung cells, (V) the stomach cells, (VI) the liver cells and spleen cells, (VII) the small intestine cells, (VIII) the colon cells, (IX) the bone cells and its supportive and connective tissue cells like malignant or benign bone tumor, e.g., malignant osteogenic sarcoma, benign osteoma, cartilage tumors; like malignant chondrosarcoma or benign chondroma; bone marrow tumors like malignant myeloma or benign eosinophilic granuloma, as well as metastatic tumors from bone tissues at other locations of the body; (X) the mouth, throat, larynx, and the esophagus cells, (XI) the urinary bladder cells and urogenital system cells of male and female like ovaries, uterus, cervix of the uterus, testes, and prostate gland, (XII) the prostate cells, (XIII) the pancreas cells, like ductal carcinoma of the pancreas; (XIV) the lymphatic tissue cells like lymphomas and other tumors of lymphoid origin, (XV) the skin cells, (XVI) the respiration and respiratory systems cells including thoracic muscles and linings, (XVII) primary or secondary cancer cells of the lymph nodes (XVIII) cells of the tongue and of the bony structures of the hard palate or sinuses, (XVIV) cells of the mouth, cheeks, neck and salivary glands, (XX) the blood vessels including the heart and their linings, (XXI) cells of the smooth or skeletal muscles and their ligaments and linings, XXII) cells of the peripheral, the autonomous, the central nervous system including the cerebellum, and/or (XXIII) adipose tissue cells. In some embodiments, a cancer cell is sarcoma or adenomas.

In some embodiments, the cancer cell is the chronic myelogenous leukemia. In some embodiments, the cancer cell is the acute lymphocytic leukemia. In some embodiments, the cancer cell is papillary thyroid carcinoma. In some embodiments, the cancer cell is lung cancer and in particular NSCLC. In some embodiments, the cancer cell is prostate cancer. In some embodiments, the cancer cell is breast cancer.

In some embodiments, the cancer cell is a lymphoid neoplasm (e.g., lymphoma). In some embodiments, the lymphoid neoplasm (e.g., lymphoma) is a B-cell neoplasm. Examples of B-cell neoplasms include, but are not limited to, precursor B-cell neoplasms (e.g., precursor B-lymphoblastic leukemia/lymphoma) and peripheral B-cell neoplasms (e.g., B-cell chronic lymphocytic leukemia/prolymphocytic leukemia/small lymphocytic lymphoma (small lymphocytic (SL) NHL), lymphoplasmacytoid lymphoma/immunocytoma, mantel cell lymphoma, follicle center lymphoma, follicular lymphoma (e.g., cytologic grades: I (small cell), II (mixed small and large cell), III (large cell) and/or subtype: diffuse and predominantly small cell type), low grade/follicular non-Hodgkin's lymphoma (NHL), intermediate grade/follicular NHL, marginal zone B-cell lymphoma (e.g., extranodal (e.g., MALT-type+/−monocytoid B cells) and/or Nodal (e.g., +/−monocytoid B cells)), splenic marginal zone lymphoma (e.g., +/−villous lymphocytes), Hairy cell leukemia, plasmacytoma/plasma cell myeloma (e.g., myeloma and multiple myeloma), diffuse large B-cell lymphoma (e.g., primary mediastinal (thymic) B-cell lymphoma), intermediate grade diffuse NHL, Burkitt's lymphoma, High-grade B-cell lymphoma, Burkitt-like, high grade immunoblastic NHL, high grade lymphoblastic NHL, high grade small non-cleaved cell NHL, bulky disease NHL, AIDS-related lymphoma, and Waldenstrom's macroglobulinemia).

In some embodiments, the lymphoid neoplasm (e.g., lymphoma) is a T-cell and/or putative NK-cell neoplasm. Examples of T-cell and/or putative NK-cell neoplasms include, but are not limited to, precursor T-cell neoplasm (precursor T-lymphoblastic lymphoma/leukemia) and peripheral T-cell and NK-cell neoplasms (e.g., T-cell chronic lymphocytic leukemia/prolymphocytic leukemia, and large granular lymphocyte leukemia (LGL) (e.g., T-cell type and/or NK-cell type), cutaneous T-cell lymphoma (e.g., mycosis fungoides/Sezary syndrome), primary T-cell lymphomas unspecified (e.g., cytological categories (e.g., medium-sized cell, mixed medium and large cell), large cell, lymphoepitheloid cell, subtype hepatosplenic γδ T-cell lymphoma, and subcutaneous panniculitic T-cell lymphoma), angioimmunoblastic T-cell lymphoma (AILD), angiocentric lymphoma, intestinal T-cell lymphoma (e.g., +/−enteropathy associated), adult T-cell lymphoma/leukemia (ATL), anaplastic large cell lymphoma (ALCL) (e.g., CD30+, T- and null-cell types), anaplastic large-cell lymphoma, and Hodgkin's like).

In some embodiments, the lymphoid neoplasm (e.g., lymphoma) is Hodgkin's disease. For example, the Hodgkin's disease may be lymphocyte predominance, nodular sclerosis, mixed cellularity, lymphocyte depletion, and/or lymphocyte-rich.

In some embodiments, the cancer cell is leukemia. In some embodiments, the leukemia is chronic leukemia. Examples of chronic leukemia include, but are not limited to, chronic myelocytic I (granulocytic) leukemia, chronic myelogenous, and chronic lymphocytic leukemia (CLL). In some embodiments, the leukemia is acute leukemia. Examples of acute leukemia include, but are not limited to, acute lymphoblastic leukemia (ALL), acute myeloid leukemia, acute lymphocytic leukemia, and acute myelocytic leukemia (e.g., myeloblastic, promyelocytic, myelomonocytic, monocytic, and erythroleukemia). In some embodiments, the cancer cell is acute lymphoblastic leukemia (ALL).

In some embodiments, the cancer cell is a liquid tumor or plasmacytoma. Plasmacytoma includes, but is not limited to, myeloma. Myeloma includes, but is not limited to, an extramedullary plasmacytoma, a solitary myeloma, and multiple myeloma. In some embodiments, the plasmacytoma is multiple myeloma. Examples of multiple myeloma include, but are not limited to, IgG multiple myeloma, IgA multiple myeloma, IgD multiple myeloma, IgE multiple myeloma, and nonsecretory multiple myeloma. In some embodiments, the multiple myeloma is IgG multiple myeloma. In some embodiments, the multiple myeloma is IgA multiple myeloma. In some embodiments, the multiple myeloma is a smoldering or indolent multiple myeloma. In some embodiments, the multiple myeloma is progressive multiple myeloma. In some embodiments, multiple myeloma may be resistant to a drug, such as, but not limited to, bortezomib, dexamethasone (Dex-), doxorubicin (Dox-), and melphalan (LR).

The cells can also be cultured cells, e.g., in vitro or ex vivo. For example, the cells are cultured in vitro in a culture medium. Alternatively, for ex vivo cultured cells, cells can be obtained from an individual, for example a healthy individual, an individual affected with a disease, an individual predisposed to a disease, or an individual who has or was expected to have a disease. Cells can be obtained, as a non-limiting example, by biopsy or other surgical means know to those skilled in the art. Cells can be present in an individual, e.g., in vivo. For the use on in vivo cells, the cell can be found in an individual and display characteristics of the disease, disorder or malignancy pathology. In some embodiments, the methods are useful in lysates (e.g., cell lysates).

The nature of the target cells, body fluids or lysates, target molecules, effector portions, and effector molecules are further discussed below in the context of methods using same. It is to be understood that the methods of the present application are not limited to these exemplary uses, and can be useful for other methods by choosing the desired effector portions, effector molecules, target molecules and target cells, body fluids or lysates.

### Methods of Detecting or Screening for Target Molecules

Provided herein are methods for detecting or screening for a target using the protein complementation regulator described herein. In another aspect, provided herein are methods for measuring the level of a target molecule using a protein complementation regulator. In some embodiments, the protein complementation regulator reduces the ability of one or more complementation molecules to interact in the absence of specific binding to a target. In some embodiments, the protein complementation regulator reduces the ability of one or more complementation molecules to interact in the absence of a target.

Provided herein are methods for the detection of a target, comprising: a) reacting under conditions that permit formation of an assembled complementation complex: (i) a first complementation molecule comprising a first probe portion and a first effector portion, (ii) a second complementation molecule comprising a second probe portion and a second effector portion, and (iii) a protein complementation regulator, wherein at least one of the probe portions binds to the target and upon binding to the target, the effector portions of the complementation molecules form an assembled complementation complex, wherein the protein complementation regulator reduces the ability of the first complementation molecule and the second complementation molecule to interact in the absence of target, and wherein the protein complementation regulator allows at least one probe portion to bind the target and upon binding of the target, the effector portions of the complementation molecules interact with each other such as to form an assembled complementation complex; and b) determining if an assembled complementation complex is formed.

Also provided herein are methods for reducing target binding-independent interaction of protein complementation molecules, comprising: a) providing: (i) a first complementation molecule comprising a first probe portion and a first effector portion; (ii) a second complementation molecule comprising a second probe portion and a second effector portion; and (iii) a protein complementation regulator, wherein at least one of the probe portions binds to the target and upon binding to the target, the effector portions of the complementation molecules form an assembled complementation complex, wherein the protein complementation regulator reduces the ability of the first complementation molecule and the second complementation molecule to interact in the absence of binding to the target, and wherein the protein complementation regulator allows at least one probe portion to bind the target and upon binding of the target, the effector portions of the complementation molecules interact with each other such as to form an assembled complementation complex; and b) allowing the components to react under conditions that permit the formation of an assembled complementation complex. In some embodiments, the method is for reducing nonspecific, target binding-independent interaction

In some embodiments of any of the methods, the first complementation molecule comprises a first probe portion and a first effector portion, wherein the first effector portion comprises a molecule (e.g., a ligand, an antibody or an aptamer) which directly or indirectly recruits a first effector molecule and the second complementation molecule comprises a second probe portion and a second effector portion, wherein the second effector portion comprises a molecule (e.g., a ligand, an antibody or an aptamer) which directly or indirectly recruits a second effector molecule.

In some embodiments of any of the methods, the first complementation molecule comprises a first probe portion and a first effector portion, wherein the first effector portion comprises a first effector molecule, and the second complementation molecule comprises a second probe portion and a second effector portion, wherein the second effector portion comprises a second effector molecule.

In some embodiments of any of the methods, the first complementation molecule comprises a first probe portion and a first effector portion, wherein the first effector portion comprises a first effector molecule, and the second complementation molecule comprises a second probe portion and a second effector portion, wherein at least one of the effector portions comprises a molecule (e.g., a ligand, an antibody or an aptamer) which directly or indirectly recruits an effector molecule.

In some embodiments of any of the methods, the effector molecule is a fluorescent protein, for example, but not limited to, green fluorescent protein (GFP), enhanced green fluorescent protein (EGFP), green-fluorescent-like proteins; yellow fluorescent protein (YFP), enhanced yellow fluorescent protein (EYFP), blue fluorescent protein (BFP), enhanced blue fluorescent protein (EBFP), cyan fluorescent protein (CFP), enhanced cyan fluorescent protein (ECFP) or a red fluorescent protein (dsRED), where one of the fragments in the reconstituted fluorescent protein contains a mature preformed chromophore. All of the above mentioned fluorescent proteins and fragments thereof that will result in a fluorescing fluorescent protein are encompassed within the methods described herein. Also encompassed are those fluorescent proteins known to those of skill in the art, and fragments and genetically engineered proteins thereof.

In some embodiments of any of the methods, the protein complementation regulator may be provided in cis or in trans with the complementation molecules. In some embodiments, the protein complementation regulator is associated with one or more of the complementation molecules. In some embodiment, the protein complementation regulator is coupled with one or more of the complementation molecules. In some embodiments, the protein complementation regulator is conjugated to one or more of the complementation molecules. In some embodiments, the protein complementation regulator is covalently linked to the complementation molecules. In some embodiments, the protein complementation regulator is covalently linked in cis to the complementation molecule and is 5′ or N-terminal to the first probe portion, between the first and second probe portion, between the first and second effector portion, or 3′ or C-terminal to the second probe portion. In some embodiments, the protein complementation regulator is covalently linked in cis to the complementation molecule and is between the first effector portion and the second effector portion. In some embodiments, the first and second effector portions are two fragments of a split aptamer capable of binding an effector molecule and the protein complementation regulator is covalently linked in cis to the complementation molecule and is between the first effector portion and the second effector portion.

In some embodiments of any of the methods, the methods are used to detect or screen for a target sample. A target sample includes, but is not limited, to target cells, target body fluids, and/or target lysates. Body fluids include but are not limited to blood, urine, cerebrospinal fluid, semen and tissue excudates. In some embodiments, the target is only present in the target sample i.e., the target is a target cell-specific molecule. In some embodiments, the methods are used to detect or screen for a target cell. In some embodiments, the methods are used to detect or screen for a target in a body fluid. In some embodiments, the methods are used to detect or screen for a target in a lysate.

In some embodiments, the presence or level of a target molecule as determined by the methods disclosed herein can be measured at different time points to assess the effectiveness of a treatment. For example, the presence or level of a target molecule as determined by the methods disclosed herein, can be measured at the beginning of treatment with a therapeutic agent and then compared to the presence or level of the target at a second time point after treatment has been initiated.

The methods of detecting or screening a target as described herein are further useful in methods of diagnosis. For example, provided herein are methods of determining whether an individual is likely responding or likely not responding to a treatment or diagnosing a disease or condition comprising: a) reacting under conditions that permit formation of an assembled complementation complex: (i) a first complementation molecule comprising a first probe portion and a first effector portion, (ii) a second complementation molecule comprising a second probe portion and a second effector portion, and (iii) a protein complementation regulator, wherein at least one of the probe portions binds to the target and upon binding to the target, the effector portions of the complementation molecules form an assembled complementation complex, wherein the protein complementation regulator reduces the ability of the first complementation molecule and the second complementation molecule to interact in the absence of target, and wherein the protein complementation regulator allows at least one probe portion to bind the target and upon binding of the target, the effector portions of the complementation molecules form an assembled complementation complex; and b) determining if an assembled complementation complex is formed, which indicates, predicts, and/or correlates that an individual is likely responding or likely not responding to a treatment and/or has or does not have a disease or condition.

Methods of Treatment and/or Preventing

Provided herein are also methods for promoting cell death in a target cell using a protein complementation regulator described herein. The method can be useful for treating diseases characterized by undesirable and/or uncontrolled cell growth, such as cancer, benign neoplasms, dysplasias, hyperplasias, as well as neoplasms showing metastatic growth or any other transformations like e.g., leukoplakias which often precede a breakout of cancer. The methods can also be useful for treating pathogenic diseases.

Provided herein are methods for treating and/or preventing a disease or disorder, comprising: a) providing: (i) an effective amount of a first complementation molecule comprising a first probe portion and a first effector portion, (ii) an effective amount of a second complementation molecule comprising a second probe portion and a second effector portion, and (iii) an effective amount of a protein complementation regulator, wherein at least one of the probe portions binds to a target associated with the disease or disorder and upon binding to the target, the effector portions of the complementation molecules form an assembled complementation complex, and wherein the protein complementation regulator reduces the ability of the first complementation molecule and the second complementation molecule to interact in the absence of binding to the target, and wherein the protein complementation regulator allows at least one probe portion to bind the target and upon binding of the target, the effector portions of the complementation molecules form an assembled complementation complex and b) allowing the components to react under conditions that permit the formation of an assembled complementation complex thereby treating and/or preventing the disease or disorder.

Ex vivo methods for treating and/or preventing disease(s) are also provided. The methods can be useful for treating diseases characterized by undesirable and/or uncontrolled cell growth, such as cancer, benign neoplasms, dysplasias, hyperplasias, as well as neoplasms showing metastatic growth or any other transformations such as leukoplakias which often precede a breakout of cancer. The methods can also be useful for treating pathogenic diseases.

In these embodiments, a population of cells is taken out from an individual prior to treating the cell with the protein complementation regulator and complementation molecules. After treatment, the population of cells can be reintroduced (e.g., transplanted) into the individual. Thus, for example, in some embodiments, there is provided a method of treating a disease in an individual, comprising: a) obtaining a population of cells from the individual, wherein the population of cells comprises at least one target cell; b) providing to (e.g., introducing into) the target cell: an effective amount of a first complementation molecule comprising a first probe portion and a first effector portion, (ii) an effective amount of a second complementation molecule comprising a second probe portion and a second effector portion, and (iii) an effective amount of a protein complementation regulator, wherein at least one of the probe portions binds to a target associated with the disease or disorder and upon binding to the target, the effector portions of the complementation molecules form an assembled complementation complex, and wherein the protein complementation regulator reduces the ability of the first complementation molecule and the second complementation molecule to interact in the absence of binding to the target, and wherein the protein complementation regulator allows at least one probe portion to bind the target and upon binding of the target, the effector portions of the complementation molecules form an assembled complementation complex.

In some embodiments, the method further comprises reintroducing (e.g., transplanting) the population of cells back into the individual. In some embodiments, the population of cells is manipulated (e.g., washed) prior to the reintroduction (e.g., transplantation).

In some embodiments of any of the methods, the first complementation molecule comprises a first probe portion and a first effector portion, wherein the first effector portion comprises a molecule (e.g., a ligand, an antibody or an aptamer) which directly or indirectly recruits a first effector molecule and the second complementation molecule comprises a second probe portion and a second effector portion, wherein the second effector portion comprises a molecule (e.g., a ligand, an antibody or an aptamer) which directly or indirectly recruits a second effector molecule.

In some embodiments of any of the methods, the first complementation molecule comprises a first probe portion and a first effector portion, wherein the first effector portion comprises a first effector molecule, and the second complementation molecule comprises a second probe portion and a second effector portion, wherein the second effector portion comprises a second effector molecule.

In some embodiments of any of the methods, the first complementation molecule comprises a first probe portion and a first effector portion, wherein the first effector portion comprises a first effector molecule, and the second complementation molecule comprises a second probe portion and a second effector portion, wherein at least one of the effector portions comprises a molecule (e.g., a ligand, an antibody or an aptamer) which directly or indirectly recruits an effector molecule.

In some embodiments, the methods described herein have reduced immunogenicity and/or are non-immunogenic.

In some embodiments of any of the methods, the protein complementation regulator may be provided in cis or in trans with the complementation molecules. In some embodiments, the protein complementation regulator is associated with one or more of the complementation molecules. In some embodiment, the protein complementation regulator is coupled with one or more of the complementation molecules. In some embodiments, the protein complementation regulator is conjugated to one or more of the complementation molecules. In some embodiments, the protein complementation regulator is covalently linked to the complementation molecules. In some embodiments, the protein complementation regulator covalently linked in cis to the complementation molecule and is 5′ or N-terminal to the first probe portion, between the first and second probe portion, between the first and second effector portion, or 3′ or C-terminal to the second probe portion. In some embodiments, the protein complementation regulator covalently linked in cis to the complementation molecule and is between the first effector portion and the second effector portion. In some embodiments, the first and second effector portions are two fragments of a split aptamer capable of binding an effector molecule and the protein complementation regulator covalently linked in cis to the complementation molecule and is between the first effector portion and the second effector portion.

In some embodiments of any of the methods, the methods are used to detect or screen for a target sample. A target sample includes, but is not limited, to target cells, target body fluids, and/or target lysates. Body fluids include but are not limited to blood, urine, cerebrospinal fluid, semen and tissue excudates. In some embodiments, the target is only present in the target sample i.e., the target is a target cell-specific molecule. In some embodiments, the methods are used to detect or screen for a target cell. In some embodiments, the methods are used to detect or screen for a target in a body fluid. In some embodiments, the methods are used to detect or screen for a target in a lysate.

In some embodiments, the target is specific to the target sample. For example, the target may be a variation of a corresponding nucleic acid in normal cells. The variation (e.g., mutation or polymorphism) may include, but is not limited to one or more of a deletion, a substitution, an insertion, a rearrangement, translocation, alternative slicing, single nucleotide polymorphism, etc. In some embodiments, a nucleic acid mutation or polymorphism will result in an altered polypeptide (e.g., missense mutation or nonsense mutation). In some embodiments, a nucleic acid mutation or polymorphism will not result in an altered polypeptide (e.g., silent mutation). For example, in some embodiments, the target cell is a cancer cell and the target nucleic acid is a nucleic acid that is specific to the cancer cell.

In some embodiments, the target is a gene or polypeptide that is expressed in a malignant cell. As an example, a target nucleic acid is the nucleic acid encoding polymorphic epithelial mucin (PEM), a component of the human milk fat globule that is expressed in cells in several body tissues and also in urine and is known to be expressed in epithelial cancer cells, notably ovarian, gastric, colorectal and pancreatic cancer cells. In such an embodiment, the target nucleic acid is a nucleic acid sequence encoding of the polymorphic epithelial mucin (PEM), and/or a target polypeptide is an antigen of PEM or cytotoxic portion of PEM. In some embodiments the nucleic acid binding motif targets PEM similar to being targeted by immunotoxins, as disclosed in WO0174905, which is incorporated herein by reference. In some embodiments, the target nucleic acid or target polypeptide can be an oncogene or an oncogenic molecule or oncogene or receptor kinase signaling molecule. In some embodiments, the target nucleic acid encodes an angiogenesis protein, for example but not limited to vascular endothelial growth factor (VEGF) or VEGF-1 or homologues thereof.

In some embodiments, the target is a DNA, RNA or polypeptide corresponding to an oncogene, namely, a mutated and/or overexpressed version of a normal gene or polypeptide that in a dominant fashion can release the cell from normal restraints on growth. Oncogenes can alone or in concert with other changes or genes, contribute to a cell's tumorigenicity. Examples of oncogenes include; gp40 (v-fms); p21 (ras); p55 (v-myc); p65 (gag-jun); pp 60 (v-src); v-abl; v-erb; v-erba; v-fos etc.

In some embodiments, the target is a BCR-ABL fusion gene. In some embodiments, the target of the BCR-ABL fusion gene is target nucleic acid. In some embodiments, the target of the BCR-ABL fusion gene is target polypeptide. For example, the fusion gene can be the result of a t(9; 22)(q34; q11) translocation. In some embodiments, the target nucleic acid is the RNA product of the BCR-ABL fusion gene. Methods of inducing cell death in cells harboring a BCR-ABL fusion gene or the corresponding RNA can be particularly useful for the treatment of cancer.

In some embodiments, the target is a TEL/AML fusion gene. In some embodiments, the target of the TEL/AML fusion gene is target nucleic acid. In some embodiments, the target of the TEL/AML fusion gene is target polypeptide. For example, the fusion gene can be the result of a t(12; 21)(p13; q22) translocation. In some embodiments, the target nucleic acid is the RNA product of the TEL/AML fusion gene. Methods of inducing cell death in cells harboring a TEL/AML fusion gene or the corresponding RNA can be particularly useful for the treatment of cancer.

In some embodiments, when the target is a chromosomal rearrangement. In some embodiments, the target chromosomal rearrangement is a target nucleic acid. In some embodiments, the target chromosomal rearrangement is target polypeptide. In some embodiments, the target nucleic acid is a chromosomal rearrangement involving the MLL gene (human chromosome 11, band q23). In some embodiments, the target nucleic acid is a chromosomal rearrangement involving the EWS gene. In some embodiments, the target nucleic acid is a chromosomal rearrangement involving the RET gene. In some embodiments, the target nucleic acid is a chromosomal rearrangement involving the FUS gene. In some embodiments, the target nucleic acid is a chromosomal rearrangement involving the TCR gene. In some embodiments, the target nucleic acid is a chromosomal rearrangement involving the IG gene. In some embodiments, the target nucleic acid is a chromosomal rearrangement involving the TMPRSS2 gene.

In some embodiments, the target is BCR-ABL p210 or BCR-ABL p190, including, but not limited to, wherein the cancer is the chronic myelogenous leukemia. In some embodiments, the target nucleic acid is MLL-AF4, MLL-ELL, MLL-AF9, MLL-ENL, or MLL-N>50, including, but not limited to, wherein the cancer is the acute lymphocytic leukemia. In some embodiments, the target nucleic acid is EWS-FLI1, EWS-ERG, EWS-WTI, EWS-ATF1, EWS-DDIT3, FUS-ERG, FUS-CREB3L, or FUS-DDIT3, including, but not limited to, wherein the cancer is sarcoma. In some embodiments, the target nucleic acid is RET-CCDC6, RET-GOLGA5, RET-KTN1, RET-NCOA4, RET-PCM1, RET-PRKAR1A, RET-TRIM24, RET-RAB61P2, RET-MBD1, TFG-NTRK1, or TPM3-NTRK1, including, but not limited to, wherein the cancer is papillary thyroid carcinoma. In some embodiments, the target nucleic acid is EML4-ALK, TGF-ALK, SLC34A2-cROS, CD74-cROS, including, but not limited to, wherein the cancer is lung cancer and in particular NSCLC. In some embodiments, the target nucleic acid is TMPRSS2-ERG, TMPRSS2-ETV1, TMPRSS2-ETV4, TMPRSS2-ETV5, SLC45A3-ETV1, SLC45A3-ETV5, HERV-K-ETV1, C15orf21-ETV1, HNRPA2V1-ETV1, KLK2-ETV4, or CANT1-ETV4, including, but not limited to, wherein the cancer is prostate cancer. In some embodiments, the target nucleic acid is ETV-NTRK3, including, but not limited to, wherein the cancer is breast cancer. See, e.g., Rabbits, Cell 137:391-395 (2009) and Lobato et al., J. of the NCI Monographs 39:58-63 (2008), which are incorporated by reference in their entirety.

In some embodiments, the target is a mutant EGFR nucleic acid or polypeptide. In some embodiments, the target is a truncation of an EGFR gene. In some embodiments, the target nucleic acid is a truncated EGFR mRNA. In some embodiments, the target is a truncated EGFR polypeptide. In some embodiments, the mutant EGFR is EGFR vIII. See, e.g., Pedersen et al. Annals of Oncology 12:745-760 (2001) and Moscatello et al. Cancer Research 55:5536-5539 (1995). Methods in cells harboring a mutant EGFR nucleic acid can be particularly useful for the treatment of cancer, for example, non-small cell lung cancer, breast cancer, ovarian cancer, brain tumor, glioma, or medulloblastoma. In some embodiments, the cancer is ovarian cancer. In some embodiments, the cancer is breast cancer.

In some embodiments, the target cell is a cancer cell and the target molecule is present at higher levels in a cancer cell. For example, in some embodiments, the target is HER2/neu or ErbB2. In some embodiments, the target HER2/neu or ErbB2 is target nucleic acid. In some embodiments, the target HER2/neu or ErbB2 is target polypeptide. In some embodiments, the target cell is breast cancer cell or ovarian cancer cell.

In some embodiments, the target is specific to a pathogen, which is characterized to cells infected with the pathogen. These include, but are not limited to, nucleic acid and/or polypeptide sequences from viral genomes such as from hepatitis type A, hepatitis type B, hepatitis type C, influenza, varicella, adenovirus, HSV-1, HSV-II, rinderpest rhinovirus, echovirus, retroviruses, rotavirus, respiratory syncytial virus, papilloma virus, papova virus, cytomegalovirus, echinovirus, abovirus, hantavirus, coxsackie virus, mumps virus, measles virus, rubella virus, polio virus, HIV-1, HIV-II, SARS, avian and/or bird flu viruses and other viruses or variants thereof. Additional pathogens include but are not limited to viruses, fungi, bacteria, parasites and other infectious organisms or molecules therefrom. In some embodiments, viruses can be selected from a group of viruses comprising of Herpes simplex virus type-1, Herpes simplex virus type-2, Cytomegalovirus, Epstein-Barr virus, Varicella-zoster virus, Human herpes virus 6, Human herpes virus 7, Human herpes virus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B. Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B. Rotavirus C, Sindbis virus, Simian immunodeficiency virus, Human T-cell Leukemia virus type-1, Hantavirus, Rubella virus, Human Immunodeficiency virus type-1, and Human Immunodeficiency virus type-2.

The methods and compositions described herein are also suitable for treating and/or preventing diseases such as cancer, which include, but are not limited to, cancers and tumor diseases of (I) the bone marrow and bone marrow derived cells (leukemias), (II) the endocrine and exocrine glands like e.g., thyroid, parathyroid, pituitary, adrenal glands, salivary glands, pancreas, (III) the breast, like e.g., benign or malignant tumors in the mammary glands of either a male or a female, the mammary ducts, adenocarcinoma, medullary carcinoma, comedo carcinoma, Paget's disease of the nipple, inflammatory carcinoma of the young woman, (IV) the lung, (V) the stomach, (VI) the liver and spleen, (VII) the small intestine, (VIII) the colon, (IX) the bone and its supportive and connective tissues like malignant or benign bone tumor, e.g., malignant osteogenic sarcoma, benign osteoma, cartilage tumors; like malignant chondrosarcoma or benign chondroma; bone marrow tumors like malignant myeloma or benign eosinophilic granuloma, as well as metastatic tumors from bone tissues at other locations of the body; (X) the mouth, throat, larynx, and the esophagus, (XI) the urinary bladder and the internal and external organs and structures of the urogenital system of male and female like ovaries, uterus, cervix of the uterus, testes, and prostate gland, (XII) the prostate, (XIII) the pancreas, like ductal carcinoma of the pancreas; (XIV) the lymphatic tissue like lymphomas and other tumors of lymphoid origin, (XV) the skin, (XVI) cancers and tumor diseases of all anatomical structures belonging to the respiration and respiratory systems including thoracic muscles and linings, (XVII) primary or secondary cancer of the lymph nodes (XVIII) the tongue and of the bony structures of the hard palate or sinuses, (XVIV) the mouth, cheeks, neck and salivary glands, (XX) the blood vessels including the heart and their linings, (XXI) the smooth or skeletal muscles and their ligaments and linings, XXII) the peripheral, the autonomous, the central nervous system including the cerebellum, and/or (XXIII) the adipose tissue. In some embodiments, a cancer is sarcoma or adenomas.

In some embodiments, the cancer is the chronic myelogenous leukemia. In some embodiments, the cancer is the acute lymphocytic leukemia. In some embodiments, the cancer is papillary thyroid carcinoma. In some embodiments, the cancer is lung cancer and in particular NSCLC. In some embodiments, the cancer is prostate cancer. In some embodiments, the cancer is breast cancer.

In some embodiments, the cancer is a lymphoid neoplasm (e.g., lymphoma). In some embodiments, the lymphoid neoplasm (e.g., lymphoma) is a B-cell neoplasm. Examples of B-cell neoplasms include, but are not limited to, precursor B-cell neoplasms (e.g., precursor B-lymphoblastic leukemia/lymphoma) and peripheral B-cell neoplasms (e.g., B-cell chronic lymphocytic leukemia/prolymphocytic leukemia/small lymphocytic lymphoma (small lymphocytic (SL) NHL), lymphoplasmacytoid lymphoma/immunocytoma, mantel cell lymphoma, follicle center lymphoma, follicular lymphoma (e.g., cytologic grades: I (small cell), II (mixed small and large cell), III (large cell) and/or subtype: diffuse and predominantly small cell type), low grade/follicular non-Hodgkin's lymphoma (NHL), intermediate grade/follicular NHL, marginal zone B-cell lymphoma (e.g., extranodal (e.g., MALT-type+/−monocytoid B cells) and/or Nodal (e.g., +/−monocytoid B cells)), splenic marginal zone lymphoma (e.g., +/−villous lymphocytes), Hairy cell leukemia, plasmacytoma/plasma cell myeloma (e.g., myeloma and multiple myeloma), diffuse large B-cell lymphoma (e.g., primary mediastinal (thymic) B-cell lymphoma), intermediate grade diffuse NHL, Burkitt's lymphoma, High-grade B-cell lymphoma, Burkitt-like, high grade immunoblastic NHL, high grade lymphoblastic NHL, high grade small non-cleaved cell NHL, bulky disease NHL, AIDS-related lymphoma, and Waldenstrom's macroglobulinemia).

In some embodiments the lymphoid neoplasm (e.g., lymphoma) is a T-cell and/or putative NK-cell neoplasm. Examples of T-cell and/or putative NK-cell neoplasms include, but are not limited to, precursor T-cell neoplasm (precursor T-lymphoblastic lymphoma/leukemia) and peripheral T-cell and NK-cell neoplasms (e.g., T-cell chronic lymphocytic leukemia/prolymphocytic leukemia, and large granular lymphocyte leukemia (LGL) (e.g., T-cell type and/or NK-cell type), cutaneous T-cell lymphoma (e.g., mycosis fungoides/Sezary syndrome), primary T-cell lymphomas unspecified (e.g., cytological categories (e.g., medium-sized cell, mixed medium and large cell), large cell, lymphoepitheloid cell, subtype hepatosplenic γδ T-cell lymphoma, and subcutaneous panniculitic T-cell lymphoma), angioimmunoblastic T-cell lymphoma (AILD), angiocentric lymphoma, intestinal T-cell lymphoma (e.g., +/−enteropathy associated), adult T-cell lymphoma/leukemia (ATL), anaplastic large cell lymphoma (ALCL) (e.g., CD30+, T- and null-cell types), anaplastic large-cell lymphoma, and Hodgkin's like).

In some embodiments, the lymphoid neoplasm (e.g., lymphoma) is Hodgkin's disease. For example, the Hodgkin's disease may be lymphocyte predominance, nodular sclerosis, mixed cellularity, lymphocyte depletion, and/or lymphocyte-rich.

In some embodiments, the cancer is leukemia. In some embodiments, the leukemia is chronic leukemia. Examples of chronic leukemia include, but are not limited to, chronic myelocytic I (granulocytic) leukemia, chronic myelogenous, and chronic lymphocytic leukemia (CLL). In some embodiments, the leukemia is acute leukemia. Examples of acute leukemia include, but are not limited to, acute lymphoblastic leukemia (ALL), acute myeloid leukemia, acute lymphocytic leukemia, and acute myelocytic leukemia (e.g., myeloblastic, promyelocytic, myelomonocytic, monocytic, and erythroleukemia). In some embodiments, the cancer is acute lymphoblastic leukemia (ALL).

In some embodiments, the cancer is a liquid tumor or plasmacytoma. Plasmacytoma includes, but is not limited to, myeloma. Myeloma includes, but is not limited to, an extramedullary plasmacytoma, a solitary myeloma, and multiple myeloma. In some embodiments, the plasmacytoma is multiple myeloma. Examples of multiple myeloma include, but are not limited to, IgG multiple myeloma, IgA multiple myeloma, IgD multiple myeloma, IgE multiple myeloma, and nonsecretory multiple myeloma. In some embodiments, the multiple myeloma is IgG multiple myeloma. In some embodiments, the multiple myeloma is IgA multiple myeloma. In some embodiments, the multiple myeloma is a smoldering or indolent multiple myeloma. In some embodiments, the multiple myeloma is progressive multiple myeloma. In some embodiments, multiple myeloma may be resistant to a drug, such as, but not limited to, bortezomib, dexamethasone (Dex-), doxorubicin (Dox-), and melphalan (LR).

The methods and compositions described herein may also be useful for treating and/or preventing pathogenic diseases. Examples of pathogens include, but are not limited to, bacteria, fungus, parasite, or virus.

Non-limiting examples of viral infections are as follows; respiratory viral infections are, for example, common cold (caused by Picornaviruses (e.g., rhinoviruses), influenza viruses or respiratory syncytial viruses), Influenza (caused by influenza A or influenza B virus), herpes virus infections (herpes simplex, herpes zoster, Epstein-Barr virus, cytomegalovirus, herpes virus 6, human herpes virus 7, or herpes virus 8 (cause of Kaposi's sarcoma in people with AIDS), central nervous system viral infections (e.g., rabies, Creutzfeldt-Jakob disease (subacute spongiform encephalopathy), progressive multifocal leukoencephalopathy (rare manifestation of polyomavirus infection of the brain caused by the JC virus), tropical spastic paraparesis (HTLV-I), arbovirus infections (e.g., Arbovirus encephalitis, yellow fever, or dengue fever), arenavirus Infections (e.g., Lymphocytic choriomeningitis), hemorrhagic fevers (e.g., Bolivian and Argentinean hemorrhagic fever and Lassa fever, Hantavirus infection, Ebola and Marburg viruses).

One example of a common virus is human immunodeficiency virus (HIV) infection, which is an infection caused by HIV-1 or HIV-II virus and results in progressive destruction of lymphocytes. This leads to acquired immunodeficiency syndrome (AIDS). Other viruses include, for example, hepatitis A, hepatitis B, hepatitis C, SARS, avian flu etc.

Other pathogen viruses include sexually transmitted (venereal) diseases, for example syphilis (caused by Treponema pallidum), gonorrhea (Neisseria gonorrhoeae), ehaneroid (Hemophilus duereyi), lymphogranuloma venereum (Chlamydia traehomatis), granuloma inguinale (Calymmatobaeterium granulomatis), nongonoeoeeal urethritis and ehlamydial eervieitis (caused by Chlamydia traehomatis, Ureaplasma urealytieum, Triehomonas vaginalis or herpes simplex virus), triehomoniasis (Triehomonas vaginalis), genital candidiasis, genital herpes, genital warts (caused by papillomaviruses), or HIV infection.

In another embodiment, a pathogen can be an infection with opportunistic pathogens, often infecting people with impaired immune system, such as for example but are not limited to nocardiosis (caused by Nocardia asteroides), aspergillosis, mucormyeosis, and cytomegalovirus infection.

The methods and compositions described herein are also suitable for treating and/or preventing diseases in need thereof. In some embodiments, the individual is an invertebrate. In some embodiments, the individual is a vertebrate. In some embodiments, the individual is a mammal. In some embodiments, the individual is a human.

The methods and compositions described herein are also suitable for treating and/or preventing diseases in an individual, wherein the individual has cancer. Cancer includes, but is not limited to, cancers and tumor diseases of (I) the bone marrow and bone marrow derived cells (leukemias), (II) the endocrine and exocrine glands like e.g., thyroid, parathyroid, pituitary, adrenal glands, salivary glands, pancreas, (III) the breast, like e.g., benign or malignant tumors in the mammary glands of either a male or a female, the mammary ducts, adenocarcinoma, medullary carcinoma, comedo carcinoma, Paget's disease of the nipple, inflammatory carcinoma of the young woman, (IV) the lung, (V) the stomach, (VI) the liver and spleen, (VII) the small intestine, (VIII) the colon, (IX) the bone and its supportive and connective tissues like malignant or benign bone tumor, e.g., malignant osteogenic sarcoma, benign osteoma, cartilage tumors; like malignant chondrosarcoma or benign chondroma; bone marrow tumors like malignant myeloma or benign eosinophilic granuloma, as well as metastatic tumors from bone tissues at other locations of the body; (X) the mouth, throat, larynx, and the esophagus, (XI) the urinary bladder and the internal and external organs and structures of the urogenital system of male and female like ovaries, uterus, cervix of the uterus, testes, and prostate gland, (XII) the prostate, (XIII) the pancreas, like ductal carcinoma of the pancreas; (XIV) the lymphatic tissue like lymphomas and other tumors of lymphoid origin, (XV) the skin, (XVI) cancers and tumor diseases of all anatomical structures belonging to the respiration and respiratory systems including thoracic muscles and linings, (XVII) primary or secondary cancer of the lymph nodes (XVIII) the tongue and of the bony structures of the hard palate or sinuses, (XVIV) the mouth, cheeks, neck and salivary glands, (XX) the blood vessels including the heart and their linings, (XXI) the smooth or skeletal muscles and their ligaments and linings, XXII) the peripheral, the autonomous, the central nervous system including the cerebellum, and/or (XXIII) the adipose tissue. In some embodiments, a cancer is sarcoma or adenomas.

In some embodiments, the individual has chronic myelogenous leukemia. In some embodiments, the individual has acute lymphocytic leukemia. In some embodiments, the individual has papillary thyroid carcinoma. In some embodiments, the individual has lung cancer and in particular NSCLC. In some embodiments, the individual has prostate cancer. In some embodiments, individual has is breast cancer.

In some embodiments, the individual has a lymphoid neoplasm (e.g., lymphoma). In some embodiments, the lymphoid neoplasm (e.g., lymphoma) is a B-cell neoplasm. Examples of B-cell neoplasms include, but are not limited to, precursor B-cell neoplasms (e.g., precursor B-lymphoblastic leukemia/lymphoma) and peripheral B-cell neoplasms (e.g., B-cell chronic lymphocytic leukemia/prolymphocytic leukemia/small lymphocytic lymphoma (small lymphocytic (SL) NHL), lymphoplasmacytoid lymphoma/immunocytoma, mantel cell lymphoma, follicle center lymphoma, follicular lymphoma (e.g., cytologic grades: I (small cell), II (mixed small and large cell), III (large cell) and/or subtype: diffuse and predominantly small cell type), low grade/follicular non-Hodgkin's lymphoma (NHL), intermediate grade/follicular NHL, marginal zone B-cell lymphoma (e.g., extranodal (e.g., MALT-type+/−monocytoid B cells) and/or Nodal (e.g., +/−monocytoid B cells)), splenic marginal zone lymphoma (e.g., +/−villous lymphocytes), Hairy cell leukemia, plasmacytoma/plasma cell myeloma (e.g., myeloma and multiple myeloma), diffuse large B-cell lymphoma (e.g., primary mediastinal (thymic) B-cell lymphoma), intermediate grade diffuse NHL, Burkitt's lymphoma, High-grade B-cell lymphoma, Burkitt-like, high grade immunoblastic NHL, high grade lymphoblastic NHL, high grade small non-cleaved cell NHL, bulky disease NHL, AIDS-related lymphoma, and Waldenstrom's macroglobulinemia).

In some embodiments, the lymphoid neoplasm (e.g., lymphoma) is a T-cell and/or putative NK-cell neoplasm. Examples of T-cell and/or putative NK-cell neoplasms include, but are not limited to, precursor T-cell neoplasm (precursor T-lymphoblastic lymphoma/leukemia) and peripheral T-cell and NK-cell neoplasms (e.g., T-cell chronic lymphocytic leukemia/prolymphocytic leukemia, and large granular lymphocyte leukemia (LGL) (e.g., T-cell type and/or NK-cell type), cutaneous T-cell lymphoma (e.g., mycosis fungoides/Sezary syndrome), primary T-cell lymphomas unspecified (e.g., cytological categories (e.g., medium-sized cell, mixed medium and large cell), large cell, lymphoepitheloid cell, subtype hepatosplenic γδ T-cell lymphoma, and subcutaneous panniculitic T-cell lymphoma), angioimmunoblastic T-cell lymphoma (AILD), angiocentric lymphoma, intestinal T-cell lymphoma (e.g., +/−enteropathy associated), adult T-cell lymphoma/leukemia (ATL), anaplastic large cell lymphoma (ALCL) (e.g., CD30+, T- and null-cell types), anaplastic large-cell lymphoma, and Hodgkin's like).

In some embodiments, the lymphoid neoplasm (e.g., lymphoma) is Hodgkin's disease. For example, the Hodgkin's disease may be lymphocyte predominance, nodular sclerosis, mixed cellularity, lymphocyte depletion, and/or lymphocyte-rich.

In some embodiments, the individual has leukemia. In some embodiments, the leukemia is chronic leukemia. Examples of chronic leukemia include, but are not limited to, chronic myelocytic I (granulocytic) leukemia, chronic myelogenous, and chronic lymphocytic leukemia (CLL). In some embodiments, the leukemia is acute leukemia. Examples of acute leukemia include, but are not limited to, acute lymphoblastic leukemia (ALL), acute myeloid leukemia, acute lymphocytic leukemia, and acute myelocytic leukemia (e.g., myeloblastic, promyelocytic, myelomonocytic, monocytic, and erythroleukemia). In some embodiments, the cancer is acute lymphoblastic leukemia (ALL).

In some embodiments, the individual has a liquid tumor or plasmacytoma. Plasmacytoma includes, but is not limited to, myeloma. Myeloma includes, but is not limited to, an extramedullary plasmacytoma, a solitary myeloma, and multiple myeloma. In some embodiments, the plasmacytoma is multiple myeloma. Examples of multiple myeloma include, but are not limited to, IgG multiple myeloma, IgA multiple myeloma, IgD multiple myeloma, IgE multiple myeloma, and nonsecretory multiple myeloma. In some embodiments, the multiple myeloma is IgG multiple myeloma. In some embodiments, the multiple myeloma is IgA multiple myeloma. In some embodiments, the multiple myeloma is a smoldering or indolent multiple myeloma. In some embodiments, the multiple myeloma is progressive multiple myeloma. In some embodiments, multiple myeloma may be resistant to a drug, such as, but not limited to, bortezomib, dexamethasone (Dex-), doxorubicin (Dox-), and melphalan (LR).

### Methods of Introduction of Molecules

Methods of introducing the molecules described herein such as the protein complementation regulator and/or complementation molecule(s) into the cell depend on the nature of the molecules. The protein complementation regulator and/or complementation molecule(s) may be directly introduced into the cell or introduced by gene delivery. For example, when the protein complementation regulator and/or complementation molecule(s) is a nucleic acid, it can be introduced into the cell by any one of the following methods: virus-based gene transfer, non-viral gene transfer, transfection, calcium phosphate, lipid-based delivery, etc. When the protein complementation regulator and/or complementation molecule(s) comprises a polypeptide portion, it can be introduced into the cell by any one of the following methods: liposome-mediated delivery, etc. In some embodiments, the protein complementation regulator and/or complementation molecule(s) can be introduced by transfection, lipofection, protoplast fusion, calcium phosphate transfection, microinjection, pressure-forced entry, naked DNA, electroporation, ballistic bombardment (e.g., gene gun), sonoporation, or complexed (e.g., conjugated or fusion) to peptide and/or polypeptide which facilitates uptake and/or internalization.

When the protein complementation regulator and/or complementation molecule(s) is used for the treatment of a disease in an individual, the complementation molecules can be administered directly into the individual to be treated in a suitable pharmaceutical composition. Suitable routes for administration include, but are not limited to, by oral, pulmonary, parenteral (e.g., intramuscular, intra-articular, intraperitoneal, intravenous (IV) or subcutaneous injection), inhalation (e.g., via a fine powder formulation or a fine mist), transdermal, nasal, vaginal, rectal, or sublingual routes of administration and can be formulated in dosage forms appropriate for each route of administration

Alternatively, the protein complementation regulator and/or complementation molecule(s) may be administered ex vivo, to cells obtained from the individual. For example, the protein complementation regulator and/or complementation molecule(s) can be used as purging agents for ex vivo cleansing of the individual's cell population containing diseased cells. Specifically, a population of cells is taken from the individual, subject to ex vivo treatment with the complementation molecules by selectively killing the target cells, and transplanted back into the individual.

Dosage of the protein complementation regulator and/or complementation molecule(s) to be administered in order to affect efficient delivery into a target cell and/or achieve a phenotypic effect correlated to the delivery is determined with reference to various parameters, including the species of the individual, the age, weight, and disease status and the particular physiological conditions requiring phenotypic alteration.

Dosage also depends upon the location of the cells to be targeted within the individual. For example, target cells of the lung may require different dosages than administration into the blood stream of an organism. The dosage of the protein complementation regulator and/or complementation molecule(s) is preferably chosen so that administration causes an effective result, as measured by molecular assays or phenotypic alteration.

Such assays include Western blot of a particular protein being administered or encoded by a transgene that has been administered, immunoprecipitation, immunocytochemistry, or other techniques known to those skilled in the art. Dosages may range from 0.1-1000 mg/kg. In some embodiment, the dosage ranges from 1 mg/kg to 100 mg/kg.

The practice described herein can be achieved by employing a number of conventional techniques of molecular biology, microbiology, recombinant DNA technology, biochemistry and immunology which are within the skill of the art. Such techniques are explained fully in the literature, see, e.g., Fritsch and Maniatis, Molecular Cloning: A Laboratory Manual, 2nd Edition (1989); Ausubel et al. (eds.), Current Protocols in Molecular Biology, (1992); incorporated herein by reference.

### Compositions and Kits

Also provided herein are compositions and kits useful for methods described herein comprising the protein complementation regulator.

The protein complementation regulator described herein can be formulated into pharmaceutical compositions. For example, the complementation molecules described herein can be formulated to be administered by various means, depending on the nature of the complementation molecule and the nature of the application or diseases. Single or multiple administrations of the compositions may be administered depending on the dosage and frequency as required and tolerated by the individual. In any event, the composition should provide a sufficient quantity of the molecules to effectively treat the individual. In some embodiments, the pharmaceutical composition comprising the protein complementation regulator further comprises one of more of the complementation molecules.

The compositions for administration, in some embodiments, will commonly comprise preloaded polymeric nanoparticles and/or cationic liposomes comprising the complementation molecule(s) in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of complementation molecules in these formulations can vary widely, and will be selected primarily based on fluid volumes, viscosities, body weight and the like in accordance with the particular mode of administration selected and the individual's needs.

Thus, a typical pharmaceutical composition for intravenous administration would be about 0.01 to 100 mg per individual per day. Dosages from 0.1 up to about 1000 mg per individual per day may be used, particularly when the drug is administered to a secluded site and not into the blood stream, such as into a tumor or an organ within which a tumor resides. Actual methods for preparing parenterally administrable compositions will be known or apparent to those skilled in the art and are described in more detail in such publications as REMINGTON′S PHARMACEUTICAL SCIENCES, 18th edition, Gennaro, A. R., Ed., (1990).

The pharmaceutical composition can be administered by any means known to persons skilled in the art. For example, some methods include pump, direct injection, topical application, or administration to an individual via intrademal, subcutaneous, intravenous, intralymphatic, intranodal, intramucosal or intramuscular administration.

Provided herein uses of the pharmaceutical composition comprising a protein complementation regulator described herein in the preparation of a drug useful in the treatment and/or prevention of cancer or a viral infection or any other disease identified by persons skilled in the art whereby the methods could be used.

In one embodiment, the protein complementation regulator and/or complementation molecules are expressed by means of inclusion bodies. There exist a large number of publications which describe the recombinant production of proteins in microorganisms/prokaryotes via the inclusion bodies route, and are any such method can be used for production of the complementation molecules by persons skilled in the art. Examples of such reviews are Misawa et al., Biopolymers 51 (1999) 297-307; Lilie, H., Curr. Opin. Biotechnol. 9 (1998) 497-501; Hockney, R. C., Trends Biotechnol. 12 (1994) 456-463.

In another embodiment, the protein complementation regulator and/or complementation molecules are produced within the cell by expression from one or more expression vectors. Methods to introduce vectors into the cell are well known by persons skilled in the art and are encompassed for use, and include viral mediated mechanisms, naked DNA mechanisms, direct DNA injection etc.

The pharmacological compositions may be used in conjunction with other treatments, for example if the complementation molecule is used for the treatment of cancer, the pharmaceutical composition may be administered for example with any other anti-cancer therapy, chemotherapy and/or with anti-angiogenic treatment. If the pharmaceutical composition is used for the treatment of a pathogen, it may be administered for example with one or more other anti-viral agents etc.

Further provided herein is a kit comprising a protein complementation regulator, wherein the protein complementation regulator reduces the ability of the first complementation molecule comprising a first probe portion and a first effector portion and a second complementation molecule comprising a second probe portion and a second effector portion to interact in the absence of binding to a target, and wherein the protein complementation regulator allows at least one probe portion to bind the target and upon binding of the target, the effector portions of the complementation molecules are capable of forming an assembled complementation complex.

In some embodiments, the kit comprising the protein complementation regulators further comprises one or more of the complementation molecules. In some embodiments, one or more of the complementation molecules and/or protein complementation regulators for use in the compositions are provided in a vial, ampules, or other container. In some embodiment, the kit further comprises instructions for use (e.g., use in methods of detecting and methods of treating and/or preventing).

The disclosures of all publications, patents, patent applications and published patent applications referred to herein by an identifying citation are hereby incorporated herein by reference in their entirety.

### Exemplar Embodiments

1. A protein complementation regulator, wherein the protein complementation regulator reduces the ability of a first complementation molecule comprising a first probe portion and a first effector portion and a second complementation molecule comprising a second probe portion and a second effector portion to interact in the absence of binding to a target, and wherein the protein complementation regulator allows at least one probe portion to bind the target and upon binding of the target, the effector portions of the complementation molecules are capable of forming an assembled complementation complex.
2. A method for the detection of a target, comprising:
a) reacting components under conditions that permit formation of an assembled complementation complex, said components comprising:
(i) a first complementation molecule comprising a first probe portion and a first effector portion,
(ii) a second complementation molecule comprising a second probe portion and a second effector portion, and
(iii) a protein complementation regulator,
wherein at least one of the probe portions binds to the target and upon binding to the target, the effector portions of the complementation molecules form an assembled complementation complex, wherein the protein complementation regulator reduces the ability of the first complementation molecule and the second complementation molecule to interact in the absence of target, and wherein the protein complementation regulator allows at least one probe portion to bind the target and upon binding of the target, the effector portions of the complementation molecules form an assembled complementation complex; and
b) determining if an assembled complementation complex is formed.
3. A method for reducing target binding-independent interaction of protein complementation molecules, comprising:
a) providing:
(i) a first complementation molecule comprising a first probe portion and a first effector portion,
(ii) a second complementation molecule comprising a second probe portion and a second effector portion, and
(iii) a protein complementation regulator,
wherein at least one of the probe portions binds to the target and upon binding to the target, the effector portions of the complementation molecules form an assembled complementation complex, wherein the protein complementation regulator reduces the ability of the first complementation molecule and the second complementation molecule to interact in the absence of binding to the target, and wherein the protein complementation regulator allows at least one probe portion to bind the target and upon binding of the target, the effector portions of the complementation molecules form an assembled complementation complex; and
b) allowing the components to react under conditions that permit the formation of an assembled complementation complex.
4. A method for treating and/or preventing a disease or disorder, comprising:
a) providing:
(i) an effective amount of a first complementation molecule comprising a first probe portion and a first effector portion,
(ii) an effective amount of a second complementation molecule comprising a second probe portion and a second effector portion, and
(iii) an effective amount of a protein complementation regulator,
wherein at least one of the probe portions binds to a target associated with the disease or disorder and upon binding to the target, the effector portions of the complementation molecules form an assembled complementation complex, wherein the protein complementation regulator reduces the ability of the first complementation molecule and the second complementation molecule to interact in the absence of binding to the target, and wherein the protein complementation regulator allows at least one probe portion to bind the target and upon binding of the target, the effector portions of the complementation molecules form an assembled complementation complex;
b) allowing the components to react under conditions that permit the formation of an assembled complementation complex thereby treating and/or preventing the disease or disorder.
5. A kit comprising a protein complementation regulator, wherein the protein complementation regulator reduces the ability of the first complementation molecule comprising a first probe portion and a first effector portion and a second complementation molecule comprising a second probe portion and a second effector portion to interact in the absence of binding to a target, and wherein the protein complementation regulator allows at least one probe portion to bind the target and upon binding of the target, the effector portions of the complementation molecules are capable of forming an assembled complementation complex.
6. The protein complementation regulator, method, or kit of any one of claims 1-5, wherein the protein complementation regulator reduces the ability of the first complementation molecule comprising the first probe portion and the first effector portion and the second complementation molecule comprising the second probe portion and the second effector portion to interact upon binding of at least one probe portion to a target compared to the ability of the first and second complementation molecules to interact in the absence of a protein complementation regulator
7. The protein complementation regulator, method, or kit of any one of claims 1-6, wherein the protein complementation regulator is a polypeptide or polynucleotide.
8. The protein complementation regulator, method, or kit of claim 7, wherein the protein complementation regulator is a polynucleotide.
9. The protein complementation regulator, method, or kit of claim 8, wherein the polynucleotide is RNA or DNA.
10. The protein complementation regulator, method, or kit of claim 7, wherein the protein complementation regulator is a polypeptide.
11. The protein complementation regulator, method, or kit of claim 10, wherein the polypeptide is an antibody or polypeptide aptamer.
12. The protein complementation regulator, method, or kit of any one of claims 1-11, wherein the protein complementation regulator is coupled to one or more of the complementation molecules.
13. The protein complementation regulator, method, or kit of claim 12, wherein the protein complementation regulator is conjugated via a covalent linkage to one or more complementation molecules.
14. The protein complementation regulator, method, or kit of any one of claim 1-13, wherein the protein complementation regulator is 5′ or N-terminal to the first probe portion, between the first and second probe portion, or 3′ or C-terminal to the second probe portion.
15. The protein complementation regulator, method, or kit of any one of claims 1-13, wherein the protein complementation regulator is between the first effector portion and second effector portion.
16. The protein complementation regulator, method, or kit of any one of claim 1-15, wherein the protein complementation regulator is a polynucleotide less than about 200 bases or base pairs in length.
17. The protein complementation regulator, method, or kit of any one of claims 1-16, wherein the protein complementation regulator reduces or inhibits tertiary interaction of the complementation molecules in the absence of binding of at least one complementation molecule to the target.
18. The protein complementation regulator, method, or kit of any one of claims 1-17, wherein the protein complementation regulator reduces or inhibits formation of the assembled complementation complex in the absence of binding of at least one complementation molecule to the target.
19. The protein complementation regulator, method, or kit of any one of claims 1-18, wherein the protein complementation regulator destabilizes formation of the assembled complementation complex in the absence of binding of at least one complementation molecule to the target.
20. The protein complementation regulator, method, or kit of any one of claims 1-19, wherein both the first and second probe portions bind the target.
21. The protein complementation regulator, method, or kit of any one of claims 1-20, wherein the target is a target nucleic acid or target polypeptide.
22. The protein complementation regulator, method, or kit of any one of claim 21, wherein the target is a target nucleic acid and the target nucleic acid is RNA or DNA.
23. The protein complementation regulator, method, or kit of any one of claims 1-22, wherein the target is a target nucleic acid and the target nucleic acid is associated with a disease or disorder.
24. The protein complementation regulator, method, or kit of any one of claims 1-23, wherein the target is a target nucleic acid and the target nucleic acid is single-stranded or double-stranded.
25. The protein complementation regulator, method, or kit of any one of any one of claims 1-24, wherein the target nucleic acid is detected in vivo or in vitro.
26. The protein complementation regulator, method, or kit of any one of any one of claims 1-25, wherein the probe portion is a nucleic acid binding motif.
27. The protein complementation regulator, method, or kit of any one of claim 26, wherein the nucleic acid binding motif is a nucleic acid binding polypeptide or a polynucleotide.
28. The protein complementation regulator, method, or kit of any one of claims 1-27, wherein the first and the second probes bind to two adjacent sequences in the target nucleic acid.
29. The protein complementation regulator, method, or kit of any one of claims 1-27, wherein the first and the second probes bind to the same sequence in the target nucleic acid.
30. The protein complementation regulator, method, or kit of any one of claims 1-20, wherein the target is one or more target polypeptides.
31. The protein complementation regulator, method, or kit of claim 30, wherein the one or more target polypeptides are a target multimer.
32. The protein complementation regulator, method, or kit of claim 31, wherein the multimer is a dimer, a trimer or a tetramer.
33. The protein complementation regulator, method, or kit of any one of claims 1-20 and 30-32, wherein the target polypeptide is detected in vivo or in vitro.
34. The protein complementation regulator, method, or kit of any one of claims 1-20 and 30-33, wherein the target polypeptide is associated with a disease or disorder.
35. The protein complementation regulator, method, or kit of any one of claims 1-34, wherein the effector portion is an effector molecule or a fragment thereof or a molecule which directly or indirectly binds an effector molecule or a fragment thereof.
36. The protein complementation regulator, method, or kit of claim 35, wherein the effector molecule is a fluorophore, a toxin, or a polypeptide.
37. The protein complementation regulator, method, or kit of claim 35, wherein the molecule which directly or indirectly binds the effector molecule or fragment thereof is a polypeptide or polynucleotide.
38. The protein complementation regulator, method, or kit of claim 37, wherein the polynucleotide is an aptamer.
39. The protein complementation regulator, method, or kit of claim 38, wherein the aptamer binds eIF4a or a fragment thereof conjugated via a covalent linkage to an effector molecule or fragment thereof.
40. The protein complementation regulator, method, or kit of any one of claims 35-39, wherein the effector molecule is a split polypeptide.

### EXAMPLES

The examples below are intended to be purely exemplary of the invention and should therefore not be considered to limit the invention in any way. The following examples and detailed description are offered by way of illustration and not by way of limitation.

### Example 1

### Construction of Protein Complementation Regulator and mRNA Target

An aptamer (5′-GGGGACCGCGCCCCACATGTGAGTGAGGCCGAAAC GTAGATTAGACAGGAGGCTCACA-3′ (SEQ ID NO: 1)) having high affinity for the recomplementation of the split fusion protein EGFP-eIF4a (Valencia-Burton et al, 2007) was modified by adding 11 nucleotide sequences (5′-ACCGCAGACTT-3 (SEQ ID NO: 2) & 5′-CTCCTCACTGG-3′, (SEQ ID NO: 3)) 5′ and 3′ of the aptamer, respectively. The complete 22 nucleotide sequence is complementary to the accessible 22 nucleotide target binding sequence (5′-CCAGTGAGGAGAAGTCTGCGGT-3′(SEQ ID NO: 4)) of rabbit beta-globin mRNA (Allawi et al, 2001). The resultant modified aptamer was then split into two probe-half aptamers (probe-half aptamer #1(P1-A1): 5′-ACCGCAGACTTGGGGACCGCGCCCCACATGTGAGTGAGGCCGAAAC-3′ (SEQ ID NO:5); probe-half aptamer #2 (A2-P2): 5′-GTAGATTAGACAGGAGGCTCACACTCCTCACTGG-3′ (SEQ ID NO: 6)). Probe-half aptamer #1 was cloned using PCR mutagenesis with primers (Integrated DNA Technologies) into the first multiple cloning site of the pETDuet-1 (Novagen®) ampicillin resistant vector between the BamH I and Not I restriction enzyme sites. Probe-half aptamer #2 was also cloned using PCR mutagenesis into the second multiple cloning site of the same pETDuet-1 vector between the Bgl I and Xho I restriction enzyme sites. Both probe-half aptamer #1 and probe-half aptamer #2 are contained within a single T7 vector transcript, which encodes 106 nucleotides upstream of probe-half aptamer #1, 155 nucleotides between probe-half aptamer #1 and probe-half aptamer #2, and approximately 160 nucleotides downstream of probe-half aptamer #2 resulting in a 501 nucleotide transcript, further referred to herein as P1-A1-R-A2-P2. As negative controls, a plasmid containing only probe-half aptamer #1 resulting in a 511 nucleotide half aptamer probe transcript, further referred to herein as P1-A1-R, or a plasmid without any probe portions or aptamer sequences resulting in a transcript with only vector sequence, further referred to herein as R, were used. Plasmids were prepared using a QIAgen® Miniprep Kit and were sequenced (Davis Sequencing).

The rabbit beta-globin target mRNA (5′-acacttgettttgacacaactgtgtttacttgcaatc ccccaaaacagacagaatggtgcatctgtccagtgaggagaagtctgeggtcactgccctgtggggcaaggtgaatgtggaagaagttggtgg tgaggccctgggcaggctgctggttgtctacccatggacccagaggttcttcgagtcctttggggacctgtcctctgcaaatgctgttatgaacaatc ctaaggtgaaggctcatggcaagaaggtgctggctgccttcagtgagggtctgagtcacctggacaacctcaaaggcacctttgctaagctgagt gaactgcactgtgacaagctgcacgtggatcctgagaacttcaggctcctgggcaacgtgctggttattgtgctgtctcatcattttggcaaagaatt cactcctcaggtgcaggctgcctatcagaaggtggtggctggtgtggccaatgccctggctcacaaataccactgagatctttttccctctgccaaa aattatggggacatcatgaagccccttgagcatctgacttctggctaataaaggaaatttattttcattgc-3′ (SEQ ID NO: 7) target sequence in bold) (Sigma-Aldrich®) was converted into cDNA using an Invitrogen™ SuperScript® III First-Strand Synthesis System. The cDNA template was cloned using PCR mutagenesis with primers (Integrated DNA Technologies) into the pRSFDuet (Novagen®) kanamycin resistant vector such that the first nucleotide of the transcript was adjacent to the last nucleotide of the promoter sequence on the vector and the last nucleotide of the rabbit beta-globin transcript was adjacent to the first nucleotide of the promoter terminator sequence resulting in a 589 nucleotide transcript. A negative control plasmid was created in the same manner and is identical to the target mRNA plasmid except that the 22 nucleotide target binding sequence (5′-CCAGTGAGGAGAAGTCTGCGGT-3′ (SEQ ID NO: 4)) was scrambled (5′-GTGTCGCTAGCGCAGAGTGGAC-3′ (SEQ ID NO: 8)).

### Example 2

### Specific Detection of Target mRNA in E. coli Cells

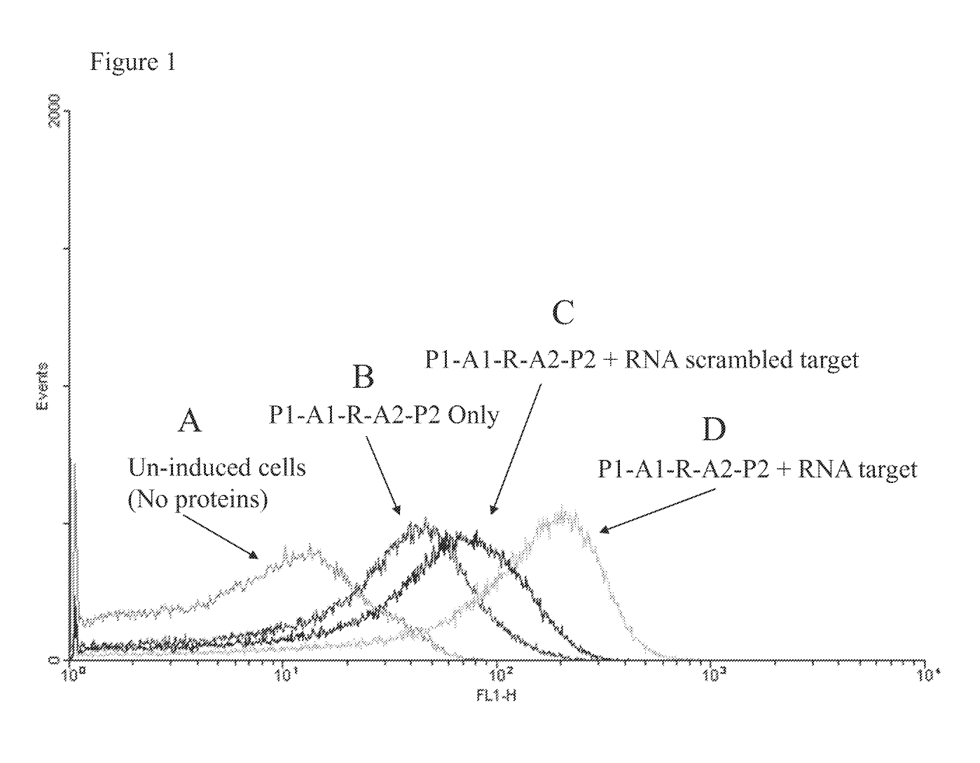

In order to assess the specificity of P1-A1-R-A2-P2 to detect target mRNA, a chloramphenicol resistant vector expressing a split fusion protein for EGFP-eIF4a as described in Valencia-Burton et al, 2007 was transformed into BL21 (DE3) E. coli cells (FIG. 1) together with the vector expressing P1-A1-R-A2-P2, the vector expressing P1-A1-R-A2-P2 and target mRNA vectors, or the vector expressing P1-A1-R-A2-P2 and negative control mRNA vectors. Un-induced cells transformed with the vector expressing split fusion protein for EGFP-eIF4a were used as a negative control. Mean fluorescent units are summarized in Table 1 and FACS histograms are shown in FIG. 1.

TABLE 1

| Sample | Mean fluorescent units (FU) |
|---|---|
| Un-induced cells | 11-12 |
| P1-A1-R-A2-P2 only | 35-42 |
| P1-A1-R-A2-P2 + RNA | 50-55 |
| scrambled target | |
| P1-A1-R-A2-P2 + RNA target | 167-170 |

Cells expressing split protein, P1-A 1-R-A2-P2 and mRNA target were about four to five times more fluorescent than cells expressing only split proteins and P1-A 1-R-A2-P2 and about three times more fluorescent than cells expressing split proteins, P1-A1-R-A2-P2 and scrambled mRNA target. These results show that P1-A1-R-A2-P2 recognizes target mRNA with high specificity and allows for the formation of a complementation complex with the split protein fragments, resulting in reassembly and fluorescence of the split protein fragments.

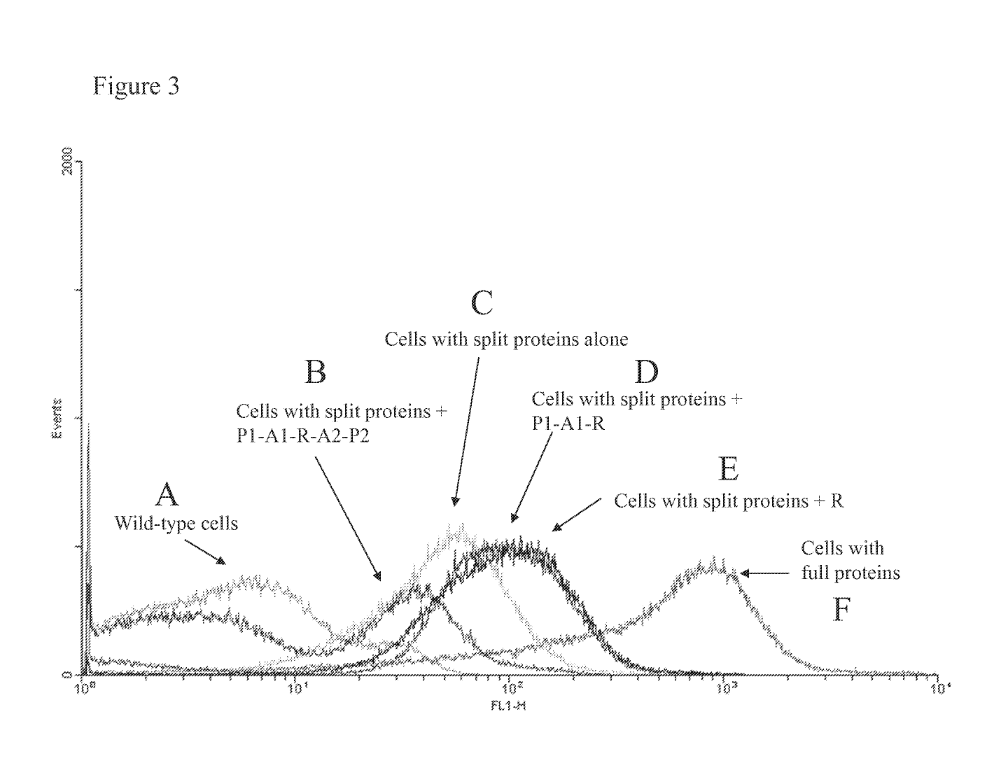

In a separate experiment, E. coli cells expressing split fusion protein for EGFP-eIF4a either alone or together with the vector expressing P1-A 1-R-A2-P2, the vector expressing only probe-half aptamer #1 (P1-A1-R) or the vector expressing no probe portions and no aptamer sequences (R) as well as wild-type E. coli cells and E. coli cells expressing full length EGFP were compared. Mean fluorescent units are summarized in Table 2 and FACS histograms are shown in FIG. 3.

TABLE 2

| Sample | Mean fluorescent units (FU) |
|---|---|
| Wild-type | 7-8 |
| Split protein + P1-A1-R-A2-P2 | 28-30 |
| Split protein alone | 75-82 |
| Split protein + P1-A1-R | 145-150 |
| Split protein + R | 128-132 |
| Full length EGFP | 750-800 |

Background fluorescence in cells expressing split protein and protein complementation regulator with two probe-half aptamers (P1-A 1-R-A2-P2) in the absence of a target mRNA (28-30) was more than two times less than background fluorescence in cells expressing only split protein in the absence of target mRNA (75-80). These results show that the presence of the protein complementation regulator significantly reduces unspecific re-assembly and fluorescence of split protein. Further, background fluorescence in cells expressing split protein and protein complementation regulator with only one probe-half aptamer (P1-A1-R) (145-150) or split protein and protein complementation regulator with no probe portions or aptamer sequences (R) (128-132), was four to five times higher than background fluorescence in cells expressing split protein P1-A1-R-A2-P2 (28-30) in the absence of a target mRNA.

All three plasmids (plasmids expressing either split protein, target mRNA or probe-half aptamer) control expression under a T7 RNA polymerase promoter and are compatible with one another in vivo. All cells were grown at 25° C. overnight in 3 ml of LB containing antibiotic (combinations of chloramphenicol, kanamycin and or ampicillin depending on vectors transformed into cells) and 1 mM IPTG unless cells were not induced, in which case no IPTG was added. Cells were collected at equal optical densities between an OD₆₀₀ of 0.4-0.6. 500 uL of cells were centrifuged and pelleted at 8000 xg for one minute and resuspended with 500 ul 1×PBS. Cell were once again centrifuged and pelleted at 8000 xg for one minute and resuspended with 500 uL of 1×PBS. Cells were then analyzed by fluorescence using flow-activated cell sorting (FACS) with a BectonDickinson FACScalibur flow cytometer.

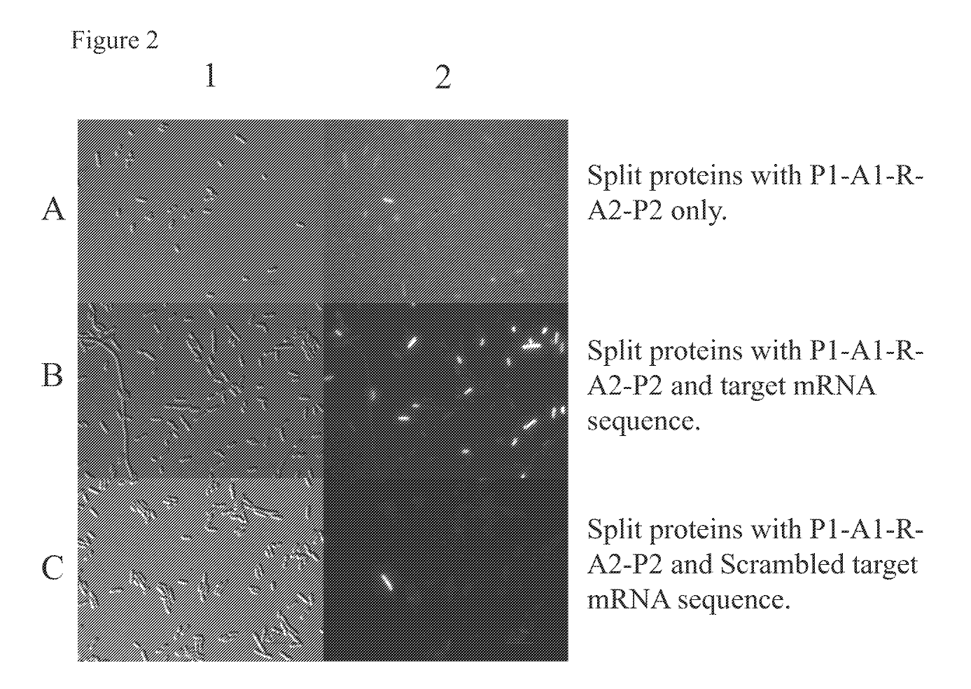

FACS analysis readings were corroborated by concurrent fluorescence microscopy using a Nikon inverted microscope Eclipse Ti-E with a Nikon CF160 optical system at 150×. FIG. 2 shows cells expressing split protein with P1-A1-R-A2-P2, split protein with P1-A 1-R-A2-P2 and target mRNA and cells expressing split protein with P1-A1-R-A2-P2 and scrambled target mRNA sequence.

### Example 3

### Detection of DNA and Protein Levels in Transformed E. coli Cells

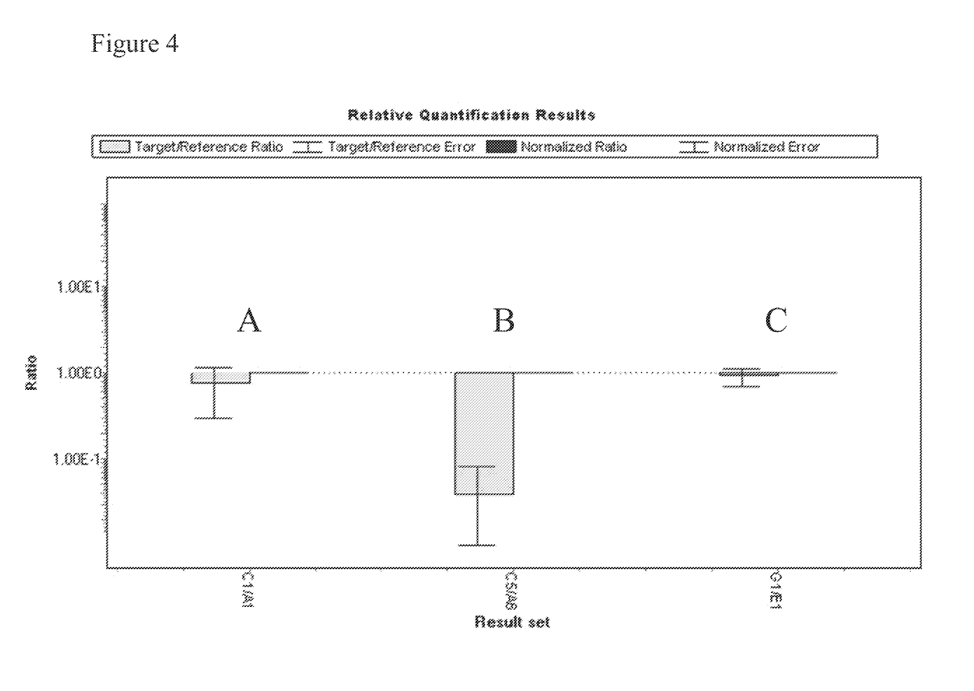

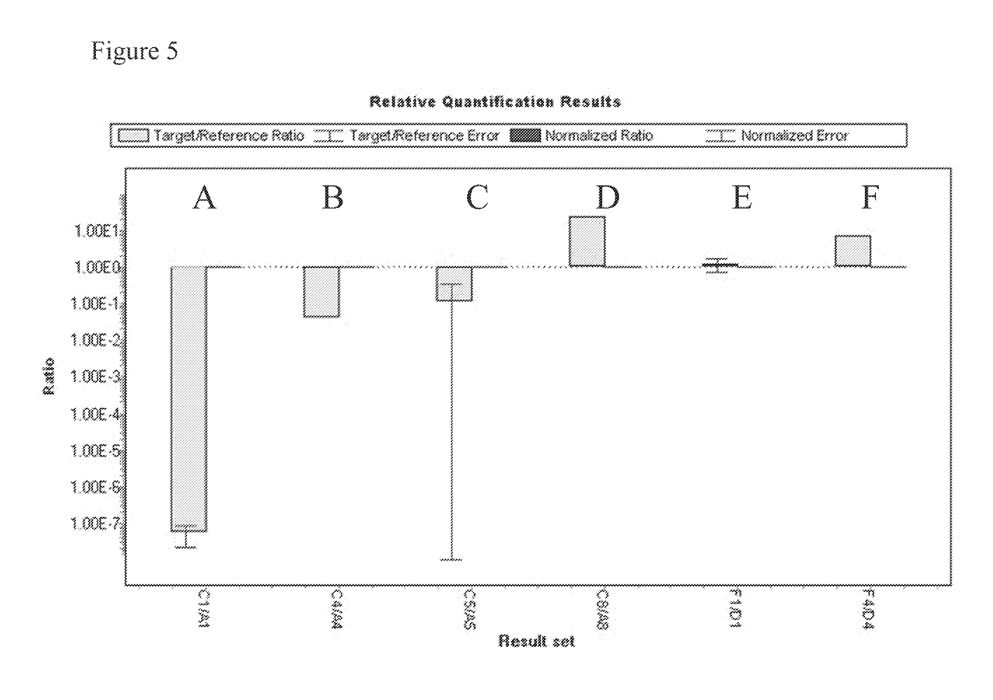

Real-time quantitative PCR analysis to detect levels of P1-A1-R-A2-P2 (FIG. 4) and levels of target mRNA (FIG. 5) was performed on cells that contained split protein together with P1-A1-R-A2-P2, split protein together with P1-A1-R-A2-P2 and target mRNA, or split protein together with P1-A1-R-A2-P2 and scrambled target mRNA as described in Example 2 and FIG. 1. RNA was prepared with a QIAgen® RNeasy® Mini Kit and turned into cDNA using an Invitrogen™ SuperScript® III First-Strand Synthesis System. For detection of target mRNA levels, control reactions without reverse transcriptase were also performed (FIGS. 5 B, D and F) Real-time primers (Integrated DNA Technologies) were used for Real-time PCR analysis using a Roche LightCycler® 480 Real-time PCR machine. Results from this analysis showed that both aptamer probe and target mRNA are expressed in all cells at the same levels as endogenous ribosomal RNA (FIGS. 4 and 5). Levels of split aptamer probes were lower in the presence of target RNA because the reverse primer used to make the aptamer amplicon targets the complementary sequence of the second aptamer probe. Since this same sequence is found on the target RNA the reverse primer is being competed away from the aptamer RNA by the target RNA sequence found in the sample (FIG. 4B).

Protein analysis will be performed on these cells by western blot analysis to confirm that protein levels are comparable.

### Example 4

### Modifications of Aptamer Probe for Optimization of Signal to Background Ratio

### Removal of Secondary Transcript

The pETDuet-1 vector expressing P1-A1-R-A2-P2 contains two T7 promoters. One promoter is upstream of the transcriptional start of the P1-A1-R-A2-P2 transcription sequence. There is a second promoter contained within the P1-A1-R-A2-P2 transcript located 64 nucleotides downstream of probe-half aptamer #1 and 75 nucleotides upstream of probe-half aptamer #2. The presence of this second promoter likely generates a secondary transcript of 275 nucleotides containing only probe-half aptamer #2. This second promoter will be abolished by PCR mutagenesis to eliminate this secondary transcript.

### Modification of Structure of Aptamer Probe Transcript

The vector sequence in the aptamer probe transcript upstream, in between and downstream of both probe-half aptamers #1 and #2 is highly structured and possesses low positional entropy values determined by RNAfold WebServer (http://rna.tbi.univie.ac.at/cgi-bin/RNAfold.cgi). This structured sequence may be either increasing or decreasing the signal to background ratio. To resolve this question unstructured sequence with a high positional entropy values will replace the structured sequence with low positional entropy by using PCR mutagenesis.

### Modification of Structure and Length of Sequence Complementary to Target

The two eleven nucleotide sequences on probe-half aptamer #1 and #2, which are complementary to the target mRNA, bind to a 22 base continuous sequence on the target mRNA. These complementary sequences will be replaced by sequences which bind to non-continuous sequences on the target mRNA, so that a gap of unbound target sequence remains between the two probe portions on the target. This gap may be sterically favorable for the reformation of the full aptamer and increase signal to background ratios. Complementary sequences on the probe-half aptamers will be modified by PCR mutagenesis to select complementary sequences for probe-half aptamers #1 and #2 which are not adjacent to each other on the target mRNA.

Length of the complementary sequence on probe-half aptamers #1 and #2 may have an effect on the signal to background ratio. To test this hypothesis various lengths of the complementary sequence on both probe-half aptamers #1 and #2 will be tested.

### Modification of Expression Levels of Target

The mRNA target is being expressed at ribosomal RNA levels off the high copy (approximately 100 copies per cell) pRSFDuet plasmid. To test the sensitivity of the P1-A 1-R-A2-P2 the mRNA target will be switched to the single copy pETcoco-2 (Novagen®) plasmid using PCR mutagenesis. By lowering the copy number of the target mRNA expression vector per cell 100 fold, it will be possible to determine if the P1-A1-R-A2-P2 will be able to detect RNAs that are expressed at low levels endogenously.

From the above description, one skilled in the art can easily ascertain the essential characteristics of the present invention, and without departing from the spirit and scope thereof, can make various changes and modifications of the invention to adapt it to various usages and conditions.

What we claim is:

1. A protein complementation regulator, wherein the protein complementation regulator reduces the ability of a first complementation molecule comprising a first probe portion and a first effector portion and a second complementation molecule comprising a second probe portion and a second effector portion to interact in the absence of binding to a target, and wherein the protein complementation regulator allows at least one probe portion to bind the target and upon binding of the target, the effector portions of the complementation molecules are capable of forming an assembled complementation complex.

2. The protein complementation regulator of claim 1, wherein the protein complementation regulator reduces the ability of the first complementation molecule comprising the first probe portion and the first effector portion and the second complementation molecule comprising the second probe portion and the second effector portion to interact upon binding of at least one probe portion to a target compared to the ability of the first and second complementation molecules to interact in the absence of a protein complementation regulator.

3. The protein complementation regulator of claim 1, wherein the protein complementation regulator is a polypeptide or polynucleotide.

4. The protein complementation regulator of claim 3, wherein the protein complementation regulator is a polynucleotide.

5. The protein complementation regulator of claim 1, wherein the protein complementation regulator is coupled to one or more of the complementation molecules.

6. The protein complementation regulator of claim 1, wherein the protein complementation regulator is 5′ or N-terminal to the first probe portion, between the first and second probe portion, or 3′ or C-terminal to the second probe portion.

7. The protein complementation regulator of claim 1, wherein the protein complementation regulator is a polynucleotide less than about 200 bases or base pairs in length.

8. The protein complementation regulator of claim 1, wherein the target is a target nucleic acid or target polypeptide.

9. The protein complementation regulator of claim 8, wherein the target is a target nucleic acid and the target nucleic acid is RNA or DNA.

10. The protein complementation regulator of claim 1, wherein the probe portion is a nucleic acid binding motif.

11. A method for the detection of a target, comprising:
a) reacting components under conditions that permit formation of an assembled complementation complex, said components comprising:
(i) a first complementation molecule comprising a first probe portion and a first effector portion,
(ii) a second complementation molecule comprising a second probe portion and a second effector portion, and
(iii) a protein complementation regulator,
wherein at least one of the probe portions binds to the target and upon binding to the target, the effector portions of the complementation molecules form an assembled complementation complex, wherein the protein complementation regulator reduces the ability of the first complementation molecule and the second complementation molecule to interact in the absence of target, and wherein the protein complementation regulator allows at least one probe portion to bind the target and upon binding of the target, the effector portions of the complementation molecules form an assembled complementation complex; and
b) determining if an assembled complementation complex is formed.

12. The method of claim 11, wherein the protein complementation regulator reduces the ability of the first complementation molecule comprising the first probe portion and the first effector portion and the second complementation molecule comprising the second probe portion and the second effector portion to interact upon binding of at least one probe portion to a target compared to the ability of the first and second complementation molecules to interact in the absence of a protein complementation regulator.

13. The method of claim 11, wherein the protein complementation regulator is a polypeptide or polynucleotide.

14. The method of claim 13, wherein the protein complementation regulator is a polynucleotide.

15. The method of claim 11, wherein the protein complementation regulator is coupled to one or more of the complementation molecules.

16. The method of claim 11, wherein the protein complementation regulator is 5′ or N-terminal to the first probe portion, between the first and second probe portion, or 3′ or C-terminal to the second probe portion.

17. The method of claim 11, wherein the protein complementation regulator is a polynucleotide less than about 200 bases or base pairs in length.

18. The method of claim 11, wherein the target is a target nucleic acid or target polypeptide.

19. The method of claim 11, wherein the probe portion is a nucleic acid binding motif.

20. A method for treating and/or preventing a disease or disorder, comprising:
a) providing:
(i) an effective amount of a first complementation molecule comprising a first probe portion and a first effector portion,
(ii) an effective amount of a second complementation molecule comprising a second probe portion and a second effector portion, and
(iii) an effective amount of a protein complementation regulator,
wherein at least one of the probe portions binds to a target associated with the disease or disorder and upon binding to the target, the effector portions of the complementation molecules form an assembled complementation complex, wherein the protein complementation regulator reduces the ability of the first complementation molecule and the second complementation molecule to interact in the absence of binding to the target, and wherein the protein complementation regulator allows at least one probe portion to bind the target and upon binding of the target, the effector portions of the complementation molecules form an assembled complementation complex;
b) allowing the components to react under conditions that permit the formation of an assembled complementation complex thereby treating and/or preventing the disease or disorder.
